(19) United States
(12) Patent Application Publication
(10) Pub. No.: US 2024/0352527 A1
(43) Pub. Date: Oct. 24, 2024

(54) METHODS AND SYSTEMS FOR DETERMINING A PREGNANCY-RELATED STATE OF A SUBJECT

(71) Applicant: Mirvie, Inc., South San Francisco, CA (US)

(72) Inventors: Maneesh JAIN, San Francisco, CA (US); Eugeni NAMSARAEV, Santa Clara, CA (US); Morten RASMUSSEN, South San Francisco, CA (US); Joan Camunas SOLER, South San Francisco, CA (US); Farooq SIDDIQUI, South San Francisco, CA (US); Mitsu REDDY, South San Francisco, CA (US); Elaine GEE, South San Francisco, CA (US); Arkady KHODURSKY, South San Francisco, CA (US); Rory NOLAN, South San Francisco, CA (US); Manfred LEE, Belmont, CA (US)

(21) Appl. No.: 18/653,375

(22) Filed: May 2, 2024

Related U.S. Application Data
(63) Continuation of application No. PCT/US2022/079235, filed on Nov. 3, 2022.
(60) Provisional application No. 63/275,726, filed on Nov. 4, 2021.
(60) Provisional application No. 63/276,809, filed on Nov. 8, 2021.
(60) Provisional application No. 63/288,044, filed on Dec. 10, 2021.

(51) Int. Cl. C12Q 1/6883; G16B 20/00; G16B 25/10; G16H 20/10; G16H 50/20; G16H 50/30

(52) U.S. Cl. CPC C12Q 1/6883; G16B 20/00; G16B 25/10; G16H 20/10; G16H 50/20; G16H 50/30; C12Q 2600/154; C12Q 2600/158

(57) ABSTRACT

The present disclosure provides methods and systems directed to cell-free identification and/or monitoring of pregnancy-related states. A method for identifying or monitoring a presence or elevated risk of a pregnancy-related state of a pregnant subject may comprise assaying a cell-free biological sample derived from said pregnant subject to detect a set of biomarkers, and analyzing the set of biomarkers with a trained algorithm to determine the presence or elevated risk of the pregnancy-related state.

### CROSS-REFERENCE

This application is a continuation of International Application No. PCT/US2022/079235, filed Nov. 3, 2022, which claims the benefit of U.S. Provisional Application No. 63/275,726, filed Nov. 4, 2021, U.S. Provisional Application No. 63/276,809, filed Nov. 8, 2021, and U.S. Provisional Application No. 63/288,044, filed Dec. 10, 2021, each of which is incorporated by reference herein in its entirety.

### BACKGROUND

Every year, about 15 million pre-term births are reported globally, and over 300,000 women die of pregnancy related complications such as hemorrhage and hypertensive disorders like preeclampsia. Pre-term birth may affect as many as about 10% of pregnancies, of which the majority are spontaneous pre-term births. Pregnancy-related complications such as pre-term birth are a leading cause of neonatal death and of complications later in life. Further, such pregnancy-related complications can cause negative health effects on maternal health.

### SUMMARY

Currently, there may be a lack of meaningful, clinically actionable diagnostic screenings or tests available for many pregnancy-related complications such as pre-term birth. Thus, to make pregnancy as safe as possible, there exists a need for rapid, accurate methods for identifying and monitoring pregnancy-related states that are non-invasive and cost-effective, toward improving maternal and fetal health.

The present disclosure provides methods, systems, and kits for identifying or monitoring pregnancy-related states by processing cell-free biological samples obtained from or derived from subjects. Cell-free biological samples (e.g., plasma samples) obtained from subjects may be analyzed to identify the pregnancy-related state (which may include, e.g., measuring a presence, absence, or relative assessment of the pregnancy-related state). Such subjects may include subjects with one or more pregnancy-related states and subjects without pregnancy-related states. Pregnancy-related states may include, for example, pre-term birth, full-term birth, gestational age, due date (e.g., due date for an unborn baby or fetus of a subject), onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In an aspect, the present disclosure provides a method for identifying a presence or elevated risk of a pregnancy-related state of a pregnant subject, comprising assaying a cell-free biological sample derived from the pregnant subject to detect a set of biomarkers, and processing the set of biomarkers with a trained algorithm or against a reference value to determine the presence or elevated risk of the pregnancy-related state among a set of at least three distinct pregnancy-related states.

In some embodiments, the pregnancy-related state is selected from the group consisting of pre-term birth, full-term birth, gestational age, due date, onset of labor, a pregnancy-related hypertensive disorder, preeclampsia, eclampsia, gestational diabetes, a congenital disorder of a fetus of the pregnant subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine/fetal growth restriction, macrosomia, a neonatal condition, and a fetal development stage or state.

In some embodiments, the pregnancy-related state is a sub-type of pre-term birth, and the at least three distinct pregnancy-related states include at least two distinct sub-types of pre-term birth. In some embodiments, the sub-type of pre-term birth is a molecular sub-type of pre-term birth, and the at least two distinct sub-types of pre-term birth include at least two distinct molecular sub-types of pre-term birth. In some embodiments, the molecular subtype of pre-term birth is selected from the group consisting of history of prior pre-term birth, spontaneous pre-term birth, ethnicity specific pre-term birth risk, and pre-term premature rupture of membrane (PPROM). In some embodiments, the molecular subtype of pre-term birth is spontaneous pre-term birth, and the set of biomarkers comprises a genomic locus associated with spontaneous pre-term birth. In some embodiments, the genomic locus associated with spontaneous pre-term birth is selected from the group consisting of genes listed in Table 1, genes listed in Table 2, genes listed in Table 3, genes corresponding to a pathway listed in Table 4, genes listed in Table 9, genes listed in Table 10, and genes listed in Table 11. In some embodiments, the spontaneous pre-term birth comprises delivery at less than 25 weeks, delivery at less than 26 weeks, delivery at less than 27 weeks, delivery at less than 28 weeks, delivery at less than 29 weeks, delivery at less than 30 weeks, delivery at less than 31 weeks, delivery at less than 32 weeks, delivery at less than 33 weeks, delivery at less than 34 weeks, delivery at less than 35 weeks, delivery at less than 36 weeks, delivery at less than 37 weeks, or delivery at less than 38 weeks. In some embodiments, the method further comprises identifying a clinical intervention for the pregnant subject based at least in part on the presence or elevated risk of the molecular sub-type of pre-term birth. In some embodiments, the clinical intervention is selected from a plurality of clinical interventions. In some embodiments, the clinical intervention comprises a drug, a supplement, a lifestyle recommendation, a cervical cerclage, a cervical pessary, or electrical contraction inhibition. In some embodiments, the drug is selected from the group consisting of progesterone, erythromycin, a tocolytic medication, a corticosteroid, a vaginal flora, and an antioxidant.

In some embodiments, the pregnancy-related state is a sub-type of preeclampsia and the at least three distinct pregnancy-related states include at least two distinct sub-types of preeclampsia. In some embodiments, the sub-type of preeclampsia is a molecular sub-type of preeclampsia, and wherein the at least two distinct sub-types of preeclampsia include at least two distinct molecular sub-types of preeclampsia. In some embodiments, the molecular subtype of preeclampsia is selected from the group consisting of history of chronic or pre-existing hypertension, presence or history of gestational hypertension, presence or history of mild preeclampsia, presence or history of severe preeclampsia, presence or history of eclampsia, and presence or history of HELLP syndrome. In some embodiments, the molecular subtype of preeclampsia is pre-term preeclampsia, and the set of biomarkers comprises a genomic locus associated with pre-term preeclampsia. In some embodiments, the genomic locus associated with pre-term preeclampsia is selected from the group consisting of genes listed in Table 5, genes listed in Table 6, genes listed in Table 7, and genes listed in Table 8. In some embodiments, the pre-term preeclampsia comprises delivery at less than 25 weeks, delivery at less than 26 weeks, delivery at less than 27 weeks, delivery at less than 28 weeks, delivery at less than 29 weeks, delivery at less than 30 weeks, delivery at less than 31 weeks, delivery at less than 32 weeks, delivery at less than 33 weeks, delivery at less than 34 weeks, delivery at less than 35 weeks, delivery at less than 36 weeks, delivery at less than 37 weeks, or delivery at less than 38 weeks. In some embodiments, the method further comprises identifying a clinical intervention for the pregnant subject based at least in part on the presence or elevated risk of the molecular subtype of preeclampsia. In some embodiments, the clinical intervention is selected from a plurality of clinical interventions. In some embodiments, the clinical intervention comprises a drug, a supplement, or a lifestyle recommendation. In some embodiments, the drug is selected from the group consisting of aspirin, progesterone, magnesium sulfate, a cholesterol medication, a heartburn medication, an angiotensin II receptor antagonist, a calcium channel blocker, a diabetes medication, and an erectile dysfunction medication.

In some embodiments, the pregnancy-related state is a sub-type of gestational diabetes, and wherein the at least three distinct pregnancy-related states include at least two distinct sub-types of gestational diabetes. In some embodiments, the sub-type of gestational diabetes is a molecular sub-type of gestational diabetes, and wherein the at least two distinct sub-types of gestational diabetes include at least two distinct molecular sub-types of gestational diabetes. In some embodiments, the set of biomarkers comprises a genomic locus associated with gestational diabetes. In some embodiments, the genomic locus associated with gestational diabetes is selected from the group consisting of PDK4, CSH1, PLAC4, TBCEL, and FBXO7. In some embodiments, the method further comprises identifying a clinical intervention for the pregnant subject based at least in part on the presence or elevated risk of the molecular sub-type of gestational diabetes. In some embodiments, the clinical intervention is selected from a plurality of clinical interventions. In some embodiments, the clinical intervention comprises a drug, a supplement, a lifestyle recommendation, a cervical cerclage, a cervical pessary, or electrical contraction inhibition.

In some embodiments, the set of biomarkers comprises at least 5 distinct genomic loci, at least 10 distinct genomic loci, at least 15 distinct genomic loci, at least 20 distinct genomic loci, at least 25 distinct genomic loci, at least 30 distinct genomic loci, at least 35 distinct genomic loci, at least 40 distinct genomic loci, at least 45 distinct genomic loci, at least 50 distinct genomic loci, at least 100 distinct genomic loci, at least 150 distinct genomic loci, or at least 200 distinct genomic loci.

In some embodiments, the assaying comprises using cell-free ribonucleic acid (cfRNA) molecules derived from the cell-free biological sample to generate transcriptomic data, using transcription products derived from the cell-free biological sample to generate transcription product data, using cell-free deoxyribonucleic acid (cfDNA) molecules derived from the cell-free biological sample to generate genomic data and/or methylation data, using proteins derived from the first cell-free biological sample to generate proteomic data, or using metabolites derived from the first cell-free biological sample to generate metabolomic data.

In some embodiments, the cell-free biological sample is selected from the group consisting of cell-free ribonucleic acid (cfRNA), cell-free deoxyribonucleic acid (cfDNA), cell-free fetal DNA (cffDNA), plasma, serum, urine, saliva, amniotic fluid, and derivatives thereof. In some embodiments, the cell-free biological sample is obtained or derived from the pregnant subject using an ethylenediaminetetraacetic acid (EDTA) collection tube, a cell-free RNA collection tube, or a cell-free deoxyribonucleic acid (DNA) collection tube. In some embodiments, the method further comprises fractionating a whole blood sample of the pregnant subject to obtain the cell-free biological sample. In some embodiments, the assaying comprises a cell-free ribonucleic acid (cfRNA) assay or a metabolomics assay. In some embodiments, the metabolomics assay comprises targeted mass spectroscopy (MS) or an immune assay. In some embodiments, the cell-free biological sample comprises cell-free ribonucleic acid (cfRNA) or urine. In some embodiments, the assaying comprises quantitative polymerase chain reaction (qPCR). In some embodiments, the assaying comprises a home use test configured to be performed in a home setting.

In some embodiments, the trained algorithm determines the presence or elevated risk of the pregnancy-related state of the pregnant subject at a sensitivity of at least about 60%, at least about 65%, at least about 70%, at least about 75%, at least about 80%, at least about 85%, at least about 90%, or at least about 95%. In some embodiments, trained algorithm determines the presence or elevated risk of the pregnancy-related state of the pregnant subject at a positive predictive value (PPV) of at least about 60%, at least about 65%, at least about 70%, at least about 75%, at least about 80%, at least about 85%, at least about 90%, or at least about 95%. In some embodiments, the trained algorithm determines the presence or elevated risk of the pregnancy-related state of the pregnant subject with an Area Under Curve (AUC) of at least about 0.60, at least about 0.65, at least about 0.70, at least about 0.75, at least about 0.80, at least about 0.85, at least about 0.90, or at least about 0.95.

In some embodiments, the pregnant subject is asymptomatic for the pregnancy-related state.

In some embodiments, the trained algorithm is trained using a first set of independent training samples associated with a presence or elevated risk of the pregnancy-related state and a second set of independent training samples associated with an absence or no elevated risk of the pregnancy-related state.

In some embodiments, the method further comprises using the trained algorithm or another trained algorithm to process a set of clinical health data of the pregnant subject to determine the presence or elevated risk of the pregnancy-related state.

In some embodiments, the method further comprises subjecting the cell-free biological sample to conditions that are sufficient to isolate, enrich, or extract a set of ribonucleic (RNA) molecules, deoxyribonucleic acid (DNA) molecules, proteins, or metabolites; and wherein the assaying comprises analyzing the set of RNA molecules, DNA molecules, proteins, or metabolites. In some embodiments, the method further comprises extracting a set of nucleic acid molecules from the cell-free biological sample, and subjecting the set of nucleic acid molecules to sequencing to generate a set of sequencing reads. In some embodiments, the sequencing comprises massively parallel sequencing. In some embodiments, the sequencing comprises nucleic acid amplification. In some embodiments, the nucleic acid amplification comprises polymerase chain reaction (PCR). In some embodiments, the sequencing comprises use of reverse transcription (RT) and polymerase chain reaction (PCR). In some embodiments, the method further comprises using probes configured to selectively enrich the set of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci. In some embodiments, the panel of the one or more genomic loci comprises a genomic locus associated with spontaneous pre-term birth, wherein the genomic locus is selected from the group consisting of genes listed in Table 1, genes listed in Table 2, genes listed in Table 3, genes corresponding to a pathway listed in Table 4, genes listed in Table 9, genes listed in Table 10, and genes listed in Table 11. In some embodiments, the panel of the one or more genomic loci comprises a genomic locus associated with pre-term preeclampsia, wherein the genomic locus is selected from the group consisting of genes listed in Table 5, genes listed in Table 6, genes listed in Table 7, and genes listed in Table 8. In some embodiments, the panel of the one or more genomic loci comprises a genomic locus associated with gestational diabetes, wherein the genomic locus is selected from the group consisting of PDK4, CSH1, PLAC4, TBCEL, and FBXO7. In some embodiments, the panel of the one or more genomic loci comprises at least 5 distinct genomic loci, at least 10 distinct genomic loci, at least 15 distinct genomic loci, at least 20 distinct genomic loci, at least 25 distinct genomic loci, at least 30 distinct genomic loci, at least 35 distinct genomic loci, at least 40 distinct genomic loci, at least 45 distinct genomic loci, at least 50 distinct genomic loci, at least 100 distinct genomic loci, at least 150 distinct genomic loci, or at least 200 distinct genomic loci.

In some embodiments, the cell-free biological sample is processed without nucleic acid isolation, enrichment, or extraction.

In some embodiments, the method further comprises generating an electronic report comprising an indication of the determined presence or elevated risk of the pregnancy-related state.

In some embodiments, the method further comprises determining a likelihood of the determination of the presence or elevated risk of the pregnancy-related state of the pregnant subject.

In some embodiments, the trained algorithm comprises a trained machine learning algorithm. In some embodiments, the trained machine learning algorithm comprises a deep learning algorithm, a support vector machine (SVM), a neural network, a Random Forest, a linear regression model, a logistic regression model, or an ANOVA model.

In some embodiments, the method further comprises processing the set of biomarkers to reduce systematic variations. In some embodiments, reducing the systematic variations comprises using residuals from multivariate linear regression to correct data residuals, performing a ComBat method based on an empirical Bayes approach, and performing a surrogate variables analysis (SVA) correction. In some embodiments, the systematic variations comprise a depth of sequencing per sample, batch effects for individual process operations, use of various raw materials, local outside temperature of sample collection, BMI of the subject, fetal fraction, fetal gestational age at sample collection, or a combination thereof.

In some embodiments, the method further comprises monitoring the presence or elevated risk of the pregnancy-related state, wherein the monitoring comprises assessing the presence or elevated risk of the pregnancy-related state of the pregnant subject at a plurality of time points, wherein the assessing is based at least on the presence or elevated risk of the pregnancy-related state determined at each of the plurality of time points. In some embodiments, a difference in the assessment of the presence or elevated risk of the pregnancy-related state of the pregnant subject among the plurality of time points is indicative of one or more clinical indications selected from the group consisting of: (i) a diagnosis of the presence or elevated risk of the pregnancy-related state of the pregnant subject, (ii) a prognosis of the presence or elevated risk of the pregnancy-related state of the pregnant subject, and (iii) an efficacy or non-efficacy of a course of treatment for treating the presence or elevated risk of the pregnancy-related state of the pregnant subject.

In some embodiments, the pregnant subject is in a first trimester of pregnancy, a second trimester of pregnancy, or a third trimester of pregnancy.

In some embodiments, the reference value is determined from pregnant subjects and/or non-pregnant subjects. In some embodiments, processing the set of biomarkers against the reference value comprises determining a difference between the set of biomarkers and the reference value.

In another aspect, the present disclosure provides a method for identifying a presence or susceptibility of a pregnancy-related state of a subject, comprising assaying transcripts and/or metabolites in a cell-free biological sample derived from the subject to detect a set of biomarkers, and analyzing the set of biomarkers with a trained algorithm to determine the presence or susceptibility of the pregnancy-related state. In some embodiments, the method comprises assaying the transcripts in the cell-free biological sample derived from the subject to detect the set of biomarkers. In some embodiments, the transcripts are assayed with nucleic acid sequencing. In some embodiments, the method comprises assaying the metabolites in the cell-free biological sample derived from the subject to detect the set of biomarkers. In some embodiments, the metabolites are assayed with a metabolomics assay.

In another aspect, the present disclosure provides a method for identifying a presence or susceptibility of a pregnancy-related state of a subject, comprising assaying a cell-free biological sample derived from the subject to detect a set of biomarkers, and analyzing the set of biomarkers with a trained algorithm to determine the presence or susceptibility of the pregnancy-related state among a set of at least three distinct pregnancy-related states (e.g., at an accuracy of at least about 80%).

In some embodiments, the pregnancy-related state is selected from the group consisting of pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, the pregnancy-related state is a sub-type of pre-term birth, and the at least three distinct pregnancy-related states include at least two distinct sub-types of pre-term birth. In some embodiments, the sub-type of pre-term birth is a molecular sub-type of pre-term birth, and the at least two distinct sub-types of pre-term birth include at least two distinct molecular sub-types of pre-term birth. In some embodiments, the distinct molecular subtypes of pre-term birth comprise a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the pregnancy-related state is a sub-type of preeclampsia, and the at least three distinct pregnancy-related states include at least two distinct sub-types of preeclampsia. In some embodiments, the distinct molecular subtypes of preeclampsia comprise a molecular subtype of preeclampsia selected from the group consisting of: presence or history of chronic or pre-existing hypertension, presence or history of gestational hypertension, presence or history of mild preeclampsia (e.g., with delivery at greater than 34 weeks gestational age), presence or history of severe preeclampsia (e.g., with delivery at less than 34 weeks gestational age), presence or history of eclampsia, and presence or history of HELLP syndrome.

In some embodiments, the method further comprises identifying a clinical intervention for the subject based at least in part on the presence or susceptibility of the pregnancy-related state. In some embodiments, the clinical intervention is selected from a plurality of clinical interventions. In some embodiments, the method further comprises determining a likelihood of the determination of the susceptibility of the pregnancy-related state of the subject, after which subject can be provided with the clinical intervention. In some embodiments, the clinical intervention comprises a pharmacological, surgical, or procedural treatment to reduce severity, delay, or eliminate the future susceptibility pregnancy-related state of the subject (e.g., aspirin for preeclampsia and steroids for pre-term birth).

In another aspect, the present disclosure provides a method comprising assaying a cell-free biological sample derived from a subject; identifying the subject as having or at risk of having preeclampsia; and upon identifying the subject as having or at risk of having preeclampsia, administering an anti-hypertensive drug to the subject.

In some embodiments, the cell-free biological sample is collected from the subject within a given gestational age interval for detection of a pregnancy-related state. In some embodiments, the given gestational age interval is within about 1 day, about 2 days, about 3 days, about 4 days, about 5 days, about 6 days about 7 days, about 8 days, about 9 days, about 10 days, about 11 days, about 12 days, about 13 days, about 14 days, about 3 weeks, or about 4 weeks from a given gestational age. In some embodiments, the given gestational age is about 0 weeks, about 1 week, about 2 weeks, about 3 weeks, about 4 weeks, about 5 weeks, about 6 weeks, about 7 weeks, about 8 weeks, about 9 weeks, about 10 weeks, about 11 week, about 12 weeks, about 13 weeks, about 14 weeks, about 15 weeks, about 16 weeks, about 17 weeks, about 18 weeks, about 19 weeks, about 20 weeks, about 21 week, about 22 weeks, about 23 weeks, about 24 weeks, about 25 weeks, about 26 weeks, about 27 weeks, about 28 weeks, about 29 weeks, about 30 weeks, about 31 week, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, or about 45 weeks. In some embodiments, the pregnancy-related state comprises one or more of: pre-term birth, onset of labor, pregnancy-related hypertensive disorders, preeclampsia, eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions, and abnormal fetal development stages or states. For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, (a) comprises (i) subjecting the cell-free biological sample to conditions that are sufficient to isolate, enrich, or extract a set of ribonucleic (RNA) molecules, deoxyribonucleic acid (DNA) molecules, transcription products (e.g., messenger RNA, transfer RNA, or ribosomal RNA), proteins (e.g., pregnancy-associated proteins corresponding to pregnancy-associated genomic loci or genes), or metabolites, and (ii) analyzing the set of RNA molecules, DNA molecules, proteins, or metabolites using the first assay to generate the first dataset. In some embodiments, the method further comprises extracting a set of nucleic acid molecules from the cell-free biological sample, and subjecting the set of nucleic acid molecules to sequencing to generate a set of sequencing reads, wherein the first dataset comprises the set of sequencing reads. In some embodiments, (b) comprises (i) subjecting the vaginal or cervical biological sample to conditions that are sufficient to isolate, enrich, or extract a population of microbes, and (ii) analyzing the population of microbes using the second assay to generate the second dataset.

In some embodiments, the sequencing is massively parallel sequencing. In some embodiments, the sequencing comprises nucleic acid amplification. In some embodiments, the nucleic acid amplification comprises polymerase chain reaction (PCR). In some embodiments, the sequencing comprises use of simultaneous reverse transcription (RT) and polymerase chain reaction (PCR). In some embodiments, the method further comprises using probes configured to selectively enrich the set of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci.

In some embodiments, the panel of the one or more genomic loci comprises a genomic locus associated with due date. In some embodiments, the panel of the one or more genomic loci comprises a genomic locus associated with gestational age. In some embodiments, the panel of the one or more genomic loci comprises a genomic locus associated with pre-term birth. In some embodiments, the panel of the one or more genomic loci comprises a genomic locus associated with preeclampsia. In some embodiments, the panel of the one or more genomic loci comprises a genomic locus associated with fetal organ development. In some embodiments, the set of biomarkers comprises a genomic locus associated with gestational diabetes mellitus.

In some embodiments, the cell-free biological sample is processed without nucleic acid isolation, enrichment, or extraction.

In some embodiments, the report is presented on a graphical user interface of an electronic device of a user. In some embodiments, the user is the subject.

In some embodiments, the method further comprises determining a likelihood of the determination of the presence or susceptibility of the pregnancy-related state of the subject.

In some embodiments, the trained algorithm comprises a supervised machine learning algorithm. In some embodiments, the supervised machine learning algorithm comprises a deep learning algorithm, a support vector machine (SVM), a neural network, or a Random Forest. In some embodiments, the trained algorithm comprises a differential expression algorithm. In some embodiments, the differential expression algorithm comprises a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, or a combination thereof.

In some embodiments, the method further comprises providing the subject with a therapeutic intervention for the presence or susceptibility of the pregnancy-related state. In some embodiments, the therapeutic intervention comprises hydroxyprogesterone caproate, a vaginal progesterone, a natural progesterone IVR product, an prostaglandin F2 alpha receptor antagonist, or a beta2-adrenergic receptor agonist.

In some embodiments, the method further comprises monitoring the presence or susceptibility of the pregnancy-related state, wherein the monitoring comprises assessing the presence or susceptibility of the pregnancy-related state of the subject at a plurality of time points, wherein the assessing is based at least on the presence or susceptibility of the pregnancy-related state determined in (d) at each of the plurality of time points.

In some embodiments, a difference in the assessment of the presence or susceptibility of the pregnancy-related state of the subject among the plurality of time points is indicative of one or more clinical indications selected from the group consisting of: (i) a diagnosis of the presence or susceptibility of the pregnancy-related state of the subject, (ii) a prognosis of the presence or susceptibility of the pregnancy-related state of the subject, and (iii) an efficacy or non-efficacy of a course of treatment for treating the presence or susceptibility of the pregnancy-related state of the subject.

In some embodiments, the method further comprises stratifying the pre-term birth by using the trained algorithm to determine a molecular sub-type of the pre-term birth from among a plurality of distinct molecular subtypes of pre-term birth. In some embodiments, the plurality of distinct molecular subtypes of pre-term birth comprises a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the method further comprises stratifying the preeclampsia by using the trained algorithm to determine a molecular sub-type of the preeclampsia from among a plurality of distinct molecular subtypes of preeclampsia comprising a molecular subtype of preeclampsia selected from the group consisting of history of chronic/pre-existing hypertension, gestational hypertension, mild preeclampsia (e.g., with delivery at >34 weeks), severe preeclampsia (e.g., with delivery at <34 weeks), eclampsia, and HELLP syndrome.

In some embodiments, the method further comprises providing the subject with a therapeutic intervention based at least in part on the risk score indicative of the risk of pre-term birth. In some embodiments, the therapeutic intervention comprises hydroxyprogesterone caproate, a vaginal progesterone, a natural progesterone IVR product, an prostaglandin F2 alpha receptor antagonist, or a beta2-adrenergic receptor agonist.

In some embodiments, the method further comprises providing the subject with a therapeutic intervention based at least in part on the risk score indicative of the risk of preeclampsia. In some embodiments, the therapeutic intervention comprises antihypertensive drug therapy (such as but not limited to hydralazine, labetalol, nifedipine, and sodium nitroprusside), management or prevention of seizures (such as but not limited to magnesium sulfate, phenytoin, and diazepam), or prevention by low-dose aspirin therapy (e.g., 100 mg per day or less) to reduce the incidence of preeclampsia.

In some embodiments, the method further comprises monitoring the risk of pre-term birth, wherein the monitoring comprises assessing the risk of pre-term birth of the subject at a plurality of time points, wherein the assessing is based at least on the risk score indicative of the risk of pre-term birth determined in (b) at each of the plurality of time points.

In some embodiments, the method further comprises monitoring the risk of preeclampsia, wherein the monitoring comprises assessing the risk of preeclampsia of the subject at a plurality of time points, wherein the assessing is based at least on the risk score indicative of the risk of preeclampsia determined in (b) at each of the plurality of time points.

In some embodiments, the method further comprises refining the risk score indicative of the risk of preeclampsia of the subject by performing one or more subsequent clinical tests for the subject, and processing results from the one or more subsequent clinical tests using a trained algorithm to determine an updated risk score indicative of the risk of preeclampsia of the subject. In some embodiments, the one or more subsequent clinical tests comprise an ultrasound imaging or a blood test. In some embodiments, the risk score comprises a likelihood of the subject having preeclampsia within a pre-determined duration of time.

In some embodiments, the method further comprises refining the risk score indicative of the risk of preeclampsia of the subject by performing one or more subsequent clinical tests for the subject, and processing results from the one or more subsequent clinical tests using a trained algorithm to determine an updated risk score indicative of the risk of preeclampsia of the subject. In some embodiments, the one or more subsequent clinical tests comprise an ultrasound imaging or a blood test. In some embodiments, the risk score comprises a likelihood of the subject having a preeclampsia within a pre-determined duration of time.

In some embodiments, the pre-determined duration of time is about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks.

In some embodiments, the method further comprises providing the subject with a therapeutic intervention for the presence or susceptibility of the pregnancy-related state. In some embodiments, therapeutic intervention comprises a progesterone treatment such as hydroxyprogesterone caproate (e.g., 17-alpha hydroxyprogesterone caproate (17-P), LPCN 1107 from Lipocine, Makena from AMAG Pharma), a vaginal progesterone, or a natural progesterone IVR product (e.g., DARE-FRT1 (JNP-0301) from Juniper Pharma); a prostaglandin F2 alpha receptor antagonist (e.g., OBE022 from ObsEva); or a beta2-adrenergic receptor agonist (e.g., bedoradrine sulfate (MN-221) from MediciNova). Therapeutic interventions may be described by, for example, “WHO Recommendations on Interventions to Improve Preterm Birth Outcomes,” ISBN 9789241508988, World Health Organization, 2015, which is hereby incorporated by reference in its entirety. In some embodiments, the method further comprises monitoring the presence or susceptibility of the pregnancy-related state, wherein the monitoring comprises assessing the presence or susceptibility of the pregnancy-related state of the subject at a plurality of time points, wherein the assessing is based at least on the presence or susceptibility of the pregnancy-related state determined in (d) at each of the plurality of time points. In some embodiments, a difference in the assessment of the presence or susceptibility of the pregnancy-related state of the subject among the plurality of time points is indicative of one or more clinical indications selected from the group consisting of: (i) a diagnosis of the presence or susceptibility of the pregnancy-related state of the subject, (ii) a prognosis of the presence or susceptibility of the pregnancy-related state of the subject, and (iii) an efficacy or non-efficacy of a course of treatment for treating the presence or susceptibility of the pregnancy-related state of the subject.

In some embodiments, the method further comprises stratifying the pre-term birth by using the trained algorithm to determine a molecular sub-type of the pre-term birth from among a plurality of distinct molecular subtypes of pre-term birth. In some embodiments, the plurality of distinct molecular subtypes of pre-term birth comprises a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the method further comprises stratifying the preeclampsia by using the trained algorithm to determine a molecular sub-type of the preeclampsia from among a plurality of distinct molecular subtypes of preeclampsia. In some embodiments, the plurality of distinct molecular subtypes of preeclampsia comprises a molecular subtype of preeclampsia selected from the group consisting of: presence or history of chronic or pre-existing hypertension, presence or history of gestational hypertension, presence or history of mild preeclampsia (e.g., with delivery greater than 34 weeks gestational age), presence or history of severe preeclampsia (with delivery less than 34 weeks gestational age), presence or history of eclampsia, and presence or history of HELLP syndrome.

In some embodiments, the method further comprises analyzing the set of biomarkers with a trained algorithm. In some embodiments, the health or physiological condition is selected from the group consisting of pre-term birth, full-term birth, gestational age, due date, onset of labor, a pregnancy-related hypertensive disorder, eclampsia, gestational diabetes, a congenital disorder of a fetus of the subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine/fetal growth restriction, macrosomia, a neonatal condition, and a fetal development stage or state. In some embodiments, the set of biomarkers comprises a genomic locus associated with due date, gestational age, pre-term birth, preeclampsia, fetal organ development, or gestational diabetes mellitus. In some embodiments, the method further comprises selecting a therapeutic intervention for the health or physiological condition of the fetus of the pregnant subject or of the pregnant subject, based at least in part on the set of biomarkers. In some embodiments, the therapeutic intervention is selected from among a plurality of therapeutic interventions. In some embodiments, the therapeutic intervention is selected based at least in part on a molecular subtype of the health or physiological condition determined based at least in part on the set of biomarkers.

In some embodiments, the therapeutic intervention is selected based on molecular sub-type of preterm birth related to collagen-containing extracellular matrix pathway and comprising drug selected from group of collagen modulating therapeutics like thrombin, Y-27632, TNF alpha and indomethacin.

In some embodiments, the therapeutic intervention is selected based on molecular sub-type of preterm birth related to extracellular matrix (ECM) pathway and comprising a therapeutic agent, wherein the therapeutic agent is an oncofetal fibronectin modulating agent. In some embodiments, the therapeutic agent is a glucocorticoid inhibitor. In some embodiments, the therapeutic agent is dexamethasone. In some embodiments, the therapeutic agent is cycloheximide.

In some embodiments, the therapeutic intervention is selected based on molecular sub-type of preterm birth related to endoplasmic reticulum (ER) lumen pathway associated with endoplasmic reticulum stress induced by oxidative stress in decidual cells and comprising drug selected from group of ER stress inhibitors 4-phenylbutyric acid (4-PBA) and tauroursodeoxycholic acid (TUDCA).

In some embodiments, the therapeutic intervention is selected based on molecular sub-type of preterm birth related inflammation pathways and comprising drug selected from group of non-specific NF-κB inhibitors, TLR4 antagonists, TNF-α biologics, CTHE (novel class of anti-inflammatory drugs): p38 MAPK inhibitors (SKF-86002, SB202190 and SB239063), IKK complex inhibitors (NBNI, parthenolide, and, TPCA-1), or TAK1 inhibitors (5z-7-oxozeaenol (OxZnl)).

In some embodiments, the therapeutic intervention is selected based on molecular sub-type of preterm birth related insulin growth-factor transport pathway and comprising a therapeutic agent selected from a group of Metformin, Insulin-like growth factor 1 (IGF-1), Insulin-Like Growth Factor Binding Protein-3 (IGFBP-3), and modulators of glucose transporters (GLUT3, GLUT8 and GLUT9). In some embodiments, the therapeutic agent is metformin. In some embodiments, the therapeutic agent is IGF-1. In some embodiments, the therapeutic agent is IGFBP-3. In some embodiments, the therapeutic agent is a modulator of GLUT3. In some embodiments, the therapeutic agent is a modulator of GLUT8. In some embodiments, the therapeutic agent is a modulator of GLUT9.

In some embodiments, the therapeutic intervention is selected based on molecular sub-type of preterm birth related metabolism of amino acids and derivatives and comprising therapeutic agent, wherein the therapeutic agent is an endogenous metabolic modulator (EMM).

In some embodiments, the health or physiological condition comprises preeclampsia. In some embodiments, the therapeutic intervention for the preeclampsia comprises a drug, a supplement, or a lifestyle recommendation. In some embodiments, the drug is selected from the group consisting of aspirin, progesterone, magnesium sulfate, a cholesterol medication (such as pravastatin), a heartburn medication (such as esomeprazole), an angiotensin II receptor antagonist (such as losartan), a calcium channel blocker (such as nifedipine), a diabetes medication (such as myo-inositol, metformin, glucovance, and liraglutide), and an erectile dysfunction medication (such as sildenafil citrate). In some embodiments, the supplement is selected from the group consisting of calcium, vitamin D, vitamin B3, and DHA. In some embodiments, the lifestyle recommendation is selected from the group consisting of exercise, nutrition counseling, meditation, stress relief, weight loss or maintenance, and improving sleep quality. In some embodiments, the therapeutic intervention for the preeclampsia is selected from a therapeutic intervention (e.g., treatment or prophylaxis) as disclosed in “WHO recommendations: Prevention and treatment of pre-eclampsia and eclampsia,” World Health Organization, ISBN 9789241548335, World Health Organization, 2011, which is incorporated by reference herein in its entirety. In some embodiments, the therapeutic intervention for the preeclampsia is selected from a therapeutic intervention (e.g., treatment or prophylaxis) as disclosed in “Summary of recommendations: Prevention and treatment of pre-eclampsia and eclampsia,” World Health Organization, WHO reference number WHO/RHR/11.30, World Health Organization, 2011, which is incorporated by reference herein in its entirety. In some embodiments, the therapeutic intervention for the preeclampsia is selected from a therapeutic intervention (e.g., treatment or prophylaxis) as disclosed in “WHO recommendations: Drug treatment for severe hypertension in pregnancy,” World Health Organization, ISBN 9789241550437, World Health Organization, 2018, which is incorporated by reference herein in its entirety.

In some embodiments, the therapeutic intervention is selected based on molecular sub-type of preeclampsia related to early placentation steps responsible for modulating the vasodilatory mediators and inhibiting vascular remodeling, platelet aggregation, and platelet adhesion and comprising drug selected from group of direct-acting vasodilators (hydralazine, minoxidil, nitrates, nitroprusside); calcium channel blockers (verapamil, diltiazem, nifedipine, amlodipine); an antagonist of the renin-angiotensin-aldosterone system (angiotensin receptor blockers, angiotensin-converting-enzyme inhibitors); Beta-2 receptor agonist (salbutamol, terbutaline); ostsynaptic alpha-1 receptor antagonist (prazosin, phenoxybenzamine, phentolamine); centrally acting alpha-2 receptor agonist (clonidine, α-methyldopa); centrally acting alpha-2 receptor agonist (clonidine, α-methyldopa); Centrally acting alpha-2 receptor agonist (clonidine, α-methyldopa).

In some embodiments, the therapeutic intervention is selected based on molecular sub-type of preeclampsia related to the molecular sub-type of PE associated with keratinocyte endothelium pathway and comprising drug selected from group of proton pump inhibitors (PPI): omeprazole, esomeprazole, pantoprazole, rabeprazole, or lansoprazole.

In some embodiments, the health or physiological condition comprises pre-term birth. In some embodiments, the therapeutic intervention for the pre-term birth comprises a drug, a supplement, a lifestyle recommendation, a cervical cerclage, a cervical pessary, or electrical contraction inhibition. In some embodiments, the drug is selected from the group consisting of progesterone, erythromycin, a tocolytic medication (such as indomethacin), a corticosteroid, a vaginal flora (such as clindamycin and metronidazole), and an antioxidant (such as N-acetylcysteine). In some embodiments, the supplement is selected from the group consisting of calcium, vitamin D, and a probiotic (such as lactobacillus). In some embodiments, the lifestyle recommendation is selected from the group consisting of exercise, nutrition counseling, meditation, stress relief, weight loss or maintenance, and improving sleep quality. In some embodiments, the therapeutic intervention for the pre-term birth is selected from a therapeutic intervention (e.g., treatment or prophylaxis) as disclosed “WHO Recommendations on Interventions to Improve Preterm Birth Outcomes,” ISBN 9789241508988, World Health Organization, 2015, which is incorporated by reference herein in its entirety.

In some embodiments, the health or physiological condition comprises gestational diabetes mellitus (GDM). In some embodiments, the therapeutic intervention for the GDM comprises a drug, a supplement, or a lifestyle recommendation. In some embodiments, the drug is selected from the group consisting of insulin and a diabetes medication (such as myo-inositol, metformin, glucovance, and liraglutide). In some embodiments, the supplement is selected from the group consisting of vitamin D, choline, probiotics, and DHA. In some embodiments, the lifestyle recommendation is selected from the group consisting of exercise, nutrition counseling, meditation, stress relief, weight loss or maintenance, and improving sleep quality. In some embodiments, the therapeutic intervention for the gestational diabetes mellitus (GDM) is selected from a therapeutic intervention (e.g., treatment or prophylaxis) as disclosed “Diagnostic criteria and classification of hyperglycemia first detected in pregnancy,” WHO reference number WHO/NMH/MND/13.2, World Health Organization, 2013, which is incorporated by reference herein in its entirety.

In some embodiments, the therapeutic intervention is selected based on molecular sub-type of gestational diabetes mellitus (GDM) related to the molecular sub-type of GDM associated placenta deterioration, placenta insufficiency, placenta failure, placenta dysfunction, premature ageing, calcification and comprising drug selected from group of sildenafil citrate, tempol ((superoxide dismutase dismutase), resveratrol, melatonin, sofalcone, statins, metformin, or [Leu27] insulin-like growth factor-II (IGF-II),

In some embodiments, the therapeutic intervention is selected based on molecular sub-type of gestational diabetes mellitus (GDM) related to the molecular sub-type of GDM associated with mediated hyperglycemic memory and comprising drug selected from group pravastatin, aminoguanidine, rosiglitazone, grape seed proanthocyanidins extracts (GSPE), hesperidin, epalrestat, pyridoxamine, telmisartan, metformin, and pioglitazone.

In some embodiments, the therapeutic intervention is selected based on molecular sub-type of gestational diabetes mellitus (GDM) related to the molecular sub-type of GDM associated with adaptive immune system and antigen-specific pathways and comprising drug selected from group azathioprine, mycophenolate mofeti, otelixizumab, teplizumab, GAD65, DiaPep277, anti-CD20 mAb, Rapamycin/IL-2, sulfonylureas, metformin, TZDs, dipeptidyl peptidase-4 Inhibitors, sodium-glucose cotransporter 2 inhibitors, diacerein, salsalate, or GLP-1 RAs.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a computer system that is programmed or otherwise configured to implement methods provided herein.

FIGS. 2A-2C show an example of receiver-operator characteristic (ROC) curves for a predictive model using differentially expressed genes identified by gene discovery (FIG. 2A), wherein the model was validated by leave-one-out cross-validation (LOOCV), including cross-validating in site 1 with a mean area-under-the-curve (AUC) for the ROC curve of 0.77 (FIG. 2B) and testing in sites 2, 3, and 4 with a mean AUC of 0.79 (FIG. 2C).

FIG. 2D shows an example of identifying pathways in top pre-term birth marker genes via gene set enrichment analysis for gene ontology and pathways for collagen-containing extracellular matrix in samples from mothers that deliver preterm.

FIG. 3 shows an example of identifying pathways in late miscarriage or early pre-term birth via gene set enrichment analysis for gene ontology and pathways for genes set related to basement membrane and endoplasmic reticulum lumen in samples from mothers that deliver before gestational age of 25 weeks.

FIG. 4 shows a distribution of 242 collected samples at gestational age of sample collection, and gestational age at delivery.

FIG. 5A shows a receiver-operator characteristic (ROC) curve for sPTB (delivery <35 weeks of GA).

FIG. 5B shows the probability assigned by the sPTB model (delivery <35 weeks of GA) to each sample. A value of 0 corresponds to a 0% probability of a sample being classified as a sPTB case, and a value of 1 corresponds to a 100% probability of a sample being classified as a sPTB case. Samples are separated by each true label (cases in orange, controls in blue).

FIG. 5C shows a receiver-operator characteristic (ROC) curve for sPTB (delivery <25 weeks of GA).

FIG. 5D shows the probability assigned by the sPTB model (delivery <25 weeks of GA) to each sample. A value of 0 corresponds to a 0% probability of a sample being classified as a sPTB case, and a value of 1 corresponds to a 100% probability of a sample being classified as a sPTB case. Samples are separated by each true label (cases in orange, controls in blue).

FIG. 6 shows the demographic and clinical data metrics for a preeclampsia observational cohort, including 2,701 healthy participants and 335 participants diagnosed with preeclampsia.

FIG. 7 shows the demographic and clinical data for a cohort with a cutoff for preeclampsia-diagnosed subjects with delivery before 38 weeks, including 2,690 healthy participants and 199 participants who were diagnosed with preeclampsia and delivered before 38 weeks of gestational age.

FIG. 8 shows the demographic and clinical data for a cohort with a cutoff for preeclampsia-diagnosed subjects with delivery before 37 weeks, including 2,780 healthy participants and 109 participants who were diagnosed with preeclampsia and delivered before 37 weeks of gestational age.

FIG. 9A shows an example of systemic variance in cell-free RNA (cfRNA) next-generation sequencing (NGS) data associated with seasonal changes for gestational- and placenta-associated genes moving in the opposite direction as the immune cellular genes.

FIG. 9B shows an example of a high correlation between prediction of outside local temperature by gene modeling and actual temperatures recorded by weather station close to the blood collection site.

FIG. 10A shows an example of systemic variance in cfRNA NGS data associated with the time of blood draw/collection.

FIG. 10B shows a quantile-quantile (QQ) plot for differential expression between early morning and afternoon groups associated with a circadian clock.

FIG. 11A shows the discovery rate for genes by Sure Independence Screening in a repeated cross validation in log 2 counts per million (CPM) reads space.

FIG. 11B shows the discovery rate for genes by Sure Independence Screening in a repeated cross validation in log 2 raw count reads space.

FIG. 12A shows features discovered by correction in multiple spaces and thresholding by Akaike information criterion (AIC) for preterm preeclampsia cases with delivery at less than 38 weeks from down sampled counts with clinical factors.

FIG. 12B shows examples of features discovered by correction in multiple spaces and thresholding by AIC for preterm preeclampsia cases with delivery at less than 37 weeks with body mass index (BMI) and blood pressure (BP) as the only clinical factors.

FIG. 13 shows an example of an area-under-the-curve (AUC) for the ROC curve values with mean at 0.83 for a model predicting preterm preeclampsia cases with deliveries at less than 37 weeks of gestation age.

FIG. 14 shows a quantile-quantile (QQ) plot for Spearman and DESeq2 differential gene expression analyses for differentially expressed genes in spontaneous preterm cases delivered before 35 weeks of gestational age. QQ plot for Spearman ranked differential gene expression is on the left, and DESeq2 differential gene expression is on the right.

FIG. 15 shows a quantile-quantile (QQ) plot for DESeq2 differential gene expression analyses for differentially expressed genes in spontaneous preterm cases delivered before 37 weeks of gestational age. QQ plot for Spearman ranked differential gene expression is on the left, and DESeq2 differential gene expression is on the right.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. A subject can be a pregnant female subject. The subject can be a woman having a fetus (or multiple fetuses) or suspected of having the fetus (or multiple fetuses). The subject can be a person that is pregnant or is suspected of being pregnant. The subject may be displaying a symptom(s) indicative of a health or physiological state or condition of the subject, such as a pregnancy-related health or physiological state or condition of the subject. As an alternative, the subject can be asymptomatic with respect to such health or physiological state or condition.

The term “pregnancy-related state,” as used herein, generally refers to any health, physiological, and/or biochemical state or condition of a subject that is pregnant or is suspected of being pregnant, or of a fetus (or multiple fetuses) of the subject. Examples of pregnancy-related states include, without limitation, pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus. In some situations, the pregnancy-related state is not associated with the health or physiological state or condition of a fetus (or multiple fetuses) of the subject.

As used herein, the term “sample,” generally refers to a biological sample obtained from or derived from one or more subjects. Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck), or a cell-free DNA collection tube (e.g., Streck). Cell-free biological samples may be derived from whole blood samples by fractionation. Biological samples or derivatives thereof may contain cells. For example, a biological sample may be a blood sample or a derivative thereof (e.g., blood collected by a collection tube or blood drops), a vaginal sample (e.g., a vaginal swab), or a cervical sample (e.g., a cervical swab).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

Every year, about 15 million pre-term births are reported globally. Pre-term birth may affect as many as about 10% of pregnancies, of which the majority are spontaneous pre-term births. Currently, there may be no meaningful, clinically actionable diagnostic screenings or tests available for many pregnancy-related complications such as pre-term birth. However, pregnancy-related complications such as pre-term birth are a leading cause of neonatal death and of complications later in life. Further, such pregnancy-related complications can cause negative health effects on maternal health. Thus, to make pregnancy as safe as possible, there exists a need for rapid, accurate methods for identifying and monitoring pregnancy-related states that are non-invasive and cost-effective, toward improving maternal and fetal health.

Current tests for prenatal care may be in inaccessible and incomplete. For cases in which pregnancies progress without pregnancy-related complications, limited methods of pregnancy monitoring may be available for a pregnancy subject, such as molecular tests, ultrasound imaging, and estimation of gestational age and/or due date using the last menstrual period. However, such monitoring methods may be complex, expensive, and unreliable. For example, molecular tests cannot predict gestational age, ultrasound imaging is expensive and best performed during the first trimester of pregnancy, and estimation of gestational age and/or due date using the last menstrual period can be unreliable. Further, for cases in which pregnancies progress with pregnancy-related complications such as risk of spontaneous pre-term delivery, the clinical utility of molecular tests, ultrasound imaging, and demographic factors may be limited. For example, molecular tests may have a limited BMI (body mass index) range, a limited gestational age and/or due date range (about 2 weeks), and a low positive predictive value (PPV); ultrasound imaging may be expensive and have low PPV and specificity; and the use of demographic factors to predict risk of pregnancy-related complications may be unreliable. Therefore, there exists an urgent clinical need for accurate and affordable non-invasive diagnostic methods for detection and monitoring of pregnancy-related states (e.g., estimation of gestational age, due date, and/or onset of labor, and prediction of pregnancy-related complications such as pre-term birth) toward clinically actionable outcomes.

The present disclosure provides methods, systems, and kits for identifying or monitoring pregnancy-related states by processing cell-free biological samples obtained from or derived from subjects (e.g., pregnancy female subjects). Cell-free biological samples (e.g., plasma samples) obtained from subjects may be analyzed to identify the pregnancy-related state (which may include, e.g., measuring a presence, absence, or quantitative assessment (e.g., risk) of the pregnancy-related state). Such subjects may include subjects with one or more pregnancy-related states and subjects without pregnancy-related states. Pregnancy-related states may include, for example, pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, and macrosomia (large fetus for gestational age). In some embodiments, pregnancy-related states are not associated with the health of a fetus. In some embodiments, pregnancy-related states include neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea) and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

### Assaying Cell-Free Biological Samples

The cell-free biological samples may be obtained or derived from a human subject (e.g., a pregnant female subject). The cell-free biological samples may be stored in a variety of storage conditions before processing, such as different temperatures (e.g., at room temperature, under refrigeration or freezer conditions, at 25° C., at 4° C., at −18° C., −20° C., or at −80° C.) or different suspensions (e.g., EDTA collection tubes, cell-free RNA collection tubes, or cell-free DNA collection tubes).

The cell-free biological sample may be obtained from a subject with a pregnancy-related state (e.g., a pregnancy-related complication), from a subject that is suspected of having a pregnancy-related state (e.g., a pregnancy-related complication), or from a subject that does not have or is not suspected of having the pregnancy-related state (e.g., a pregnancy-related complication). The pregnancy-related state may comprise a pregnancy-related complication, such as pre-term birth, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development). The pregnancy-related state may comprise a full-term birth, normal fetal development stages or states (e.g., normal fetal organ function or development), or absence of a pregnancy-related complication (e.g., pre-term birth, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development)). The pregnancy-related state may comprise a quantitative assessment of pregnancy such as gestational age (e.g., measured in days, weeks or months) or due date (e.g., expressed as a predicted or estimated calendar date or range of calendar dates). The pregnancy-related state may comprise a quantitative assessment of a pregnancy-related complication such as a likelihood, a susceptibility, or a risk (e.g., expressed as a probability, a relative probability, an odds ratio, or a risk score or risk index) of the pregnancy-related complication (e.g., pre-term birth, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development)). For example, the pregnancy-related state may comprise a likelihood or susceptibility of an onset of labor in the future (e.g., within about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

The cell-free biological sample may be taken before and/or after treatment of a subject with the pregnancy-related complication. Cell-free biological samples may be obtained from a subject during a treatment or a treatment regime. Multiple cell-free biological samples may be obtained from a subject to monitor the effects of the treatment over time. The cell-free biological sample may be taken from a subject known or suspected of having a pregnancy-related state (e.g., pregnancy-related complication) for which a definitive positive or negative diagnosis is not available via clinical tests. The sample may be taken from a subject suspected of having a pregnancy-related complication. The cell-free biological sample may be taken from a subject experiencing unexplained symptoms, such as fatigue, nausea, weight loss, aches and pains, weakness, or bleeding. The cell-free biological sample may be taken from a subject having explained symptoms. The cell-free biological sample may be taken from a subject at risk of developing a pregnancy-related complication due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

The cell-free biological sample may contain one or more analytes capable of being assayed, such as cell-free ribonucleic acid (cfRNA) molecules suitable for assaying to generate transcriptomic data, using transcription products (e.g., messenger RNA, transfer RNA, or ribosomal RNA) derived from the cell-free biological sample to generate transcription product data, cell-free deoxyribonucleic acid (cfDNA) molecules suitable for assaying to generate genomic data and/or methylation data, proteins (e.g., pregnancy-associated proteins corresponding to pregnancy-associated genomic loci or genes) suitable for assaying to generate proteomic data, metabolites suitable for assaying to generate metabolomic data, or a mixture or combination thereof. One or more such analytes (e.g., cfRNA molecules, cfDNA molecules, proteins, or metabolites) may be isolated or extracted from one or more cell-free biological samples of a subject for downstream assaying using one or more suitable assays.

After obtaining a cell-free biological sample from the subject, the cell-free biological sample may be processed to generate datasets indicative of a pregnancy-related state of the subject. For example, a presence, absence, or quantitative assessment of nucleic acid molecules of the cell-free biological sample at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins (e.g., corresponding to pregnancy-associated genomic loci or genes), and/or metabolome data comprising quantitative measures of a panel of pregnancy-related state-associated metabolites may be indicative of a pregnancy-related state. Processing the cell-free biological sample obtained from the subject may comprise (i) subjecting the cell-free biological sample to conditions that are sufficient to isolate, enrich, or extract a plurality of nucleic acid molecules, proteins (e.g., pregnancy-associated proteins corresponding to pregnancy-associated genomic loci or genes), and/or metabolites, and (ii) assaying the plurality of nucleic acid molecules, proteins, and/or metabolites to generate the dataset.

In some embodiments, a plurality of nucleic acid molecules is extracted from the cell-free biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the cell-free biological sample by a variety of methods, such as a FastDNA Kit protocol from MP Biomedicals, a QIAamp DNA cell-free biological mini kit from Qiagen, or a cell-free biological DNA isolation kit protocol from Norgen Biotek. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with pregnancy-related states. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

RNA or DNA molecules isolated or extracted from a cell-free biological sample may be tagged, e.g., with identifiable tags, to allow for multiplexing of a plurality of samples. Any number of RNA or DNA samples may be multiplexed. For example a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial cell-free biological samples. For example, a plurality of cell-free biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the pregnancy-related state. For example, the sequence reads may be aligned to one or more reference genomes (e.g., a genome of one or more species such as a human genome). The aligned sequence reads may be quantified at one or more genomic loci to generate the datasets indicative of the pregnancy-related state. For example, quantification of sequences corresponding to a plurality of genomic loci associated with pregnancy-related states may generate the datasets indicative of the pregnancy-related state.

The cell-free biological sample may be processed without any nucleic acid extraction. For example, the pregnancy-related state may be identified or monitored in the subject by using probes configured to selectively enrich nucleic acid (e.g., RNA or DNA) molecules corresponding to the plurality of pregnancy-related state-associated genomic loci. The probes may be nucleic acid primers. The probes may have sequence complementarity with nucleic acid sequences from one or more of the plurality of pregnancy-related state-associated genomic loci or genomic regions. The plurality of pregnancy-related state-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct pregnancy-related state-associated genomic loci or genomic regions. The plurality of pregnancy-related state-associated genomic loci or genomic regions may comprise one or more members (e.g., 1, 2, 3, 4, 5, 6, 7, 8, 9, 10, 11, 12, 13, 14, 15, 16, 17, 18, 19, 20, about 25, about 30, about 35, about 40, about 45, about 50, about 55, about 60, about 65, about 70, about 75, about 80, or more) selected from the group consisting of ACTB, ADAM12, ALPP, ANXA3, APLF, ARGI, AVPRIA, CAMP, CAPN6, CD180, CGA, CGB, CLCN3, CPVL, CSH1, CSH2, CSHL1, CYP3A7, DAPP1, DCX, DEFA4, DGCR14, ELANE, ENAH, EPB42, FABP1, FAM212B-AS1, FGA, FGB, FRMD4B, FRZB, FSTL3, GH2, GNAZ, HAL, HSD17B1, HSD3B1, HSPB8, Immune, ITIH2, KLF9, KNG1, KRT8, LGALS14, LTF, LYPLAL1, MAP3K7CL, MEF2C, MMD, MMP8, MOB1B, NFATC2, OTC, P2RY12, PAPPA, PGLYRP1, PKHD1L1, PKHD1L1, PLAC1, PLAC4, POLE2, PPBP, PSG1, PSG4, PSG7, PTGER3, RAB11A, RAB27B, RAPIGAP, RGS18, RPL23AP7, S100A8, S100A9, S100P, SERPINA7, SLC2A2, SLC38A4, SLC4A1, TBCID15, VCAN, VGLL1, B3GNT2, COL24A1, CXCL8, and PTGS2. The pregnancy-related state-associated genomic loci or genomic regions may be associated with gestational age, pre-term birth, due date, onset of labor, or other pregnancy-related states or complications, such as the genomic loci described by, for example, Ngo et al. (“Noninvasive blood tests for fetal development predict gestational age and preterm delivery,” Science, 360 (6393), pp. 1133-1136, 8 Jun. 2018), which is hereby incorporated by reference in its entirety.

The probes may be nucleic acid molecules (e.g., RNA or DNA) having sequence complementarity with nucleic acid sequences (e.g., RNA or DNA) of the one or more genomic loci (e.g., pregnancy-related state-associated genomic loci). These nucleic acid molecules may be primers or enrichment sequences. The assaying of the cell-free biological sample using probes that are selective for the one or more genomic loci (e.g., pregnancy-related state-associated genomic loci) may comprise use of array hybridization (e.g., microarray-based), polymerase chain reaction (PCR), or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECTR), and CRISPR-mediated analog multi-event recording apparatus (CAMERA)), and laser transmission spectroscopy (LTS).

The assay readouts may be quantified at one or more genomic loci (e.g., pregnancy-related state-associated genomic loci) to generate the data indicative of the pregnancy-related state. For example, quantification of array hybridization or polymerase chain reaction (PCR) corresponding to a plurality of genomic loci (e.g., pregnancy-related state-associated genomic loci) may generate data indicative of the pregnancy-related state. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

In some embodiments, multiple assays are used to process cell-free biological samples of a subject. For example, a first assay may be used to process a first cell-free biological sample obtained or derived from the subject to generate a first dataset; and based at least in part on the first dataset, a second assay different from the first assay may be used to process a second cell-free biological sample obtained or derived from the subject to generate a second dataset indicative of the pregnancy-related state. The first assay may be used to screen or process cell-free biological samples of a set of subjects, while the second or subsequent assays may be used to screen or process cell-free biological samples of a smaller subset of the set of subjects. The first assay may have a low cost and/or a high sensitivity of detecting one or more pregnancy-related states (e.g., pregnancy-related complication), that is amenable to screening or processing cell-free biological samples of a relatively large set of subjects. The second assay may have a higher cost and/or a higher specificity of detecting one or more pregnancy-related states (e.g., pregnancy-related complication), that is amenable to screening or processing cell-free biological samples of a relatively small set of subjects (e.g., a subset of the subjects screened using the first assay). The second assay may generate a second dataset having a specificity (e.g., for one or more pregnancy-related states such as pregnancy-related complications) greater than the first dataset generated using the first assay. As an example, one or more cell-free biological samples may be processed using a cfRNA assay on a large set of subjects and subsequently a metabolomics assay on a smaller subset of subjects, or vice versa. The smaller subset of subjects may be selected based at least in part on the results of the first assay.

Alternatively, multiple assays may be used to simultaneously process cell-free biological samples of a subject. For example, a first assay may be used to process a first cell-free biological sample obtained or derived from the subject to generate a first dataset indicative of the pregnancy-related state; and a second assay different from the first assay may be used to process a second cell-free biological sample obtained or derived from the subject to generate a second dataset indicative of the pregnancy-related state. Any or all of the first dataset and the second dataset may then be analyzed to assess the pregnancy-related state of the subject. For example, a single diagnostic index or diagnosis score can be generated based on a combination of the first dataset and the second dataset. As another example, separate diagnostic indexes or diagnosis scores can be generated based on the first dataset and the second dataset.

The cell-free biological samples may be processed to identify a set of biomarker RNA transcripts that are indicative of a set of corresponding biomarker proteins (e.g., pregnancy-associated proteins corresponding to pregnancy-associated genomic loci or genes), pathways, and/or metabolites. For example, a given biomarker RNA transcript may be expected to be translated into a corresponding given biomarker protein or a gene regulator for a corresponding given biomarker protein. Therefore, identifying a presence or absence of the given biomarker RNA transcript in a biological sample may be indicative of a presence or absence of a corresponding biomarker protein. As another example, a given biomarker RNA transcript may be expected to correlate with a corresponding given pathway. Therefore, identifying a presence or absence of the given biomarker RNA transcript in a biological sample may be indicative of a presence or absence of the corresponding pathway activity. As another example, a given biomarker RNA transcript may be expected to correlate with a corresponding given biomarker metabolite. Therefore, identifying a presence or absence of the given biomarker RNA transcript in a biological sample may be indicative of a presence or absence of the corresponding biomarker metabolite. In some embodiments, the set of corresponding biomarker proteins, pathways, and/or metabolites comprises pregnancy-related state-associated proteins (e.g., corresponding to pregnancy-associated genomic loci or genes), pathways, and/or metabolites. In some embodiments, the set of corresponding biomarker proteins, pathways, and/or metabolites comprises placental proteins, pathways, and/or metabolites. For example, identifying a presence or absence of the PAPPA gene may be indicative of a presence or absence of the PAPPA protein analog.

The cell-free biological samples may be processed using a metabolomics assay. For example, a metabolomics assay can be used to identify a quantitative measure (e.g., indicative of a presence, absence, or relative amount) of each of a plurality of pregnancy-related state-associated metabolites in a cell-free biological sample of the subject. The metabolomics assay may be configured to process cell-free biological samples such as a blood sample or a urine sample (or derivatives thereof) of the subject. A quantitative measure (e.g., indicative of a presence, absence, or relative amount) of pregnancy-related state-associated metabolites in the cell-free biological sample may be indicative of one or more pregnancy-related states. The metabolites in the cell-free biological sample may be produced (e.g., as an end product or a byproduct) as a result of one or more metabolic pathways corresponding to pregnancy-related state-associated genes. Assaying one or more metabolites of the cell-free biological sample may comprise isolating or extracting the metabolites from the cell-free biological sample. The metabolomics assay may be used to generate datasets indicative of the quantitative measure (e.g., indicative of a presence, absence, or relative amount) of each of a plurality of pregnancy-related state-associated metabolites in the cell-free biological sample of the subject.

The metabolomics assay may analyze a variety of metabolites in the cell-free biological sample, such as small molecules, lipids, amino acids, peptides, nucleotides, hormones and other signaling molecules, cytokines, minerals and elements, polyphenols, fatty acids, dicarboxylic acids, alcohols and polyols, alkanes and alkenes, keto acids, glycolipids, carbohydrates, hydroxy acids, purines, prostanoids, catecholamines, acyl phosphates, phospholipids, cyclic amines, amino ketones, nucleosides, glycerolipids, aromatic acids, retinoids, amino alcohols, pterins, steroids, carnitines, leukotrienes, indoles, porphyrins, sugar phosphates, coenzyme A derivatives, glucuronides, ketones, sugar phosphates, inorganic ions and gases, sphingolipids, bile acids, alcohol phosphates, amino acid phosphates, aldehydes, quinones, pyrimidines, pyridoxals, tricarboxylic acids, acyl glycines, cobalamin derivatives, lipoamides, biotin, and polyamines.

The metabolomics assay may comprise, for example, one or more of: mass spectroscopy (MS), targeted MS, gas chromatography (GC), high performance liquid chromatography (HPLC), capillary electrophoresis (CE), nuclear magnetic resonance (NMR) spectroscopy, ion-mobility spectrometry, Raman spectroscopy, electrochemical assay, or immune assay.

The cell-free biological samples may be processed using a methylation-specific assay. For example, a methylation-specific assay can be used to identify a quantitative measure (e.g., indicative of a presence, absence, or relative amount) of methylation each of a plurality of pregnancy-related state-associated genomic loci in a cell-free biological sample of the subject. The methylation-specific assay may be configured to process cell-free biological samples such as a blood sample or a urine sample (or derivatives thereof) of the subject. A quantitative measure (e.g., indicative of a presence, absence, or relative amount) of methylation of pregnancy-related state-associated genomic loci in the cell-free biological sample may be indicative of one or more pregnancy-related states. The methylation-specific assay may be used to generate datasets indicative of the quantitative measure (e.g., indicative of a presence, absence, or relative amount) of methylation of each of a plurality of pregnancy-related state-associated genomic loci in the cell-free biological sample of the subject.

The methylation-specific assay may comprise, for example, one or more of: a methylation-aware sequencing (e.g., using bisulfite treatment), pyrosequencing, methylation-sensitive single-strand conformation analysis (MS-SSCA), high-resolution melting analysis (HRM), methylation-sensitive single-nucleotide primer extension (MS-SnuPE), base-specific cleavage/MALDI-TOF, microarray-based methylation assay, methylation-specific PCR, targeted bisulfite sequencing, oxidative bisulfite sequencing, mass spectroscopy-based bisulfite sequencing, or reduced representation bisulfite sequence (RRBS).

The cell-free biological samples may be processed using a proteomics assay. For example, a proteomics assay can be used to identify a quantitative measure (e.g., indicative of a presence, absence, or relative amount) of each of a plurality of pregnancy-related state-associated proteins (e.g., corresponding to pregnancy-associated genomic loci or genes) or polypeptides in a cell-free biological sample of the subject. The proteomics assay may be configured to process cell-free biological samples such as a blood sample or a urine sample (or derivatives thereof) of the subject. A quantitative measure (e.g., indicative of a presence, absence, or relative amount) of pregnancy-related state-associated proteins (e.g., corresponding to pregnancy-associated genomic loci or genes) or polypeptides in the cell-free biological sample may be indicative of one or more pregnancy-related states. The proteins or polypeptides in the cell-free biological sample may be produced (e.g., as an end product, an intermediate product, or a byproduct) as a result of one or more biochemical pathways corresponding to pregnancy-related state-associated genes. Assaying one or more proteins or polypeptides of the cell-free biological sample may comprise isolating or extracting the proteins or polypeptides from the cell-free biological sample. The proteomics assay may be used to generate datasets indicative of the quantitative measure (e.g., indicative of a presence, absence, or relative amount) of each of a plurality of pregnancy-related state-associated proteins or polypeptides in the cell-free biological sample of the subject.

The proteomics assay may analyze a variety of proteins (e.g., pregnancy-associated proteins corresponding to pregnancy-associated genomic loci or genes) or polypeptides in the cell-free biological sample, such as proteins made under different cellular conditions (e.g., development, cellular differentiation, or cell cycle). The proteomics assay may comprise, for example, one or more of: an antibody-based immunoassay, an Edman degradation assay, a mass spectrometry-based assay (e.g., matrix-assisted laser desorption/ionization (MALDI) and electrospray ionization (ESI)), a top-down proteomics assay, a bottom-up proteomics assay, a mass spectrometric immunoassay (MSIA), a stable isotope standard capture with anti-peptide antibodies (SISCAPA) assay, a fluorescence two-dimensional differential gel electrophoresis (2-D DIGE) assay, a quantitative proteomics assay, a protein microarray assay, or a reverse-phased protein microarray assay. The proteomics assay may detect post-translational modifications of proteins or polypeptides (e.g., phosphorylation, ubiquitination, methylation, acetylation, glycosylation, oxidation, and nitrosylation). The proteomics assay may identify or quantify one or more proteins or polypeptides from a database (e.g., Human Protein Atlas, PeptideAtlas, and UniProt).

### Kits

The present disclosure provides kits for identifying or monitoring a pregnancy-related state of a subject. A kit may comprise probes for identifying a quantitative measure (e.g., indicative of a presence, absence, or relative amount) of sequences at each of a plurality of pregnancy-related state-associated genomic loci in a cell-free biological sample of the subject. A quantitative measure (e.g., indicative of a presence, absence, or relative amount) of sequences at each of a plurality of pregnancy-related state-associated genomic loci in the cell-free biological sample may be indicative of one or more pregnancy-related states. The probes may be selective for the sequences at the plurality of pregnancy-related state-associated genomic loci in the cell-free biological sample. A kit may comprise instructions for using the probes to process the cell-free biological sample to generate datasets indicative of a quantitative measure (e.g., indicative of a presence, absence, or relative amount) of sequences at each of the plurality of pregnancy-related state-associated genomic loci in a cell-free biological sample of the subject.

The probes in the kit may be selective for the sequences at the plurality of pregnancy-related state-associated genomic loci in the cell-free biological sample. The probes in the kit may be configured to selectively enrich nucleic acid (e.g., RNA or DNA) molecules corresponding to the plurality of pregnancy-related state-associated genomic loci. The probes in the kit may be nucleic acid primers. The probes in the kit may have sequence complementarity with nucleic acid sequences from one or more of the plurality of pregnancy-related state-associated genomic loci or genomic regions. The plurality of pregnancy-related state-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct pregnancy-related state-associated genomic loci or genomic regions.

The instructions in the kit may comprise instructions to assay the cell-free biological sample using the probes that are selective for the sequences at the plurality of pregnancy-related state-associated genomic loci in the cell-free biological sample. These probes may be nucleic acid molecules (e.g., RNA or DNA) having sequence complementarity with nucleic acid sequences (e.g., RNA or DNA) from one or more of the plurality of pregnancy-related state-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the cell-free biological sample to generate datasets indicative of a quantitative measure (e.g., indicative of a presence, absence, or relative amount) of sequences at each of the plurality of pregnancy-related state-associated genomic loci in the cell-free biological sample. A quantitative measure (e.g., indicative of a presence, absence, or relative amount) of sequences at each of a plurality of pregnancy-related state-associated genomic loci in the cell-free biological sample may be indicative of one or more pregnancy-related states.

The instructions in the kit may comprise instructions to measure and interpret assay readouts, which may be quantified at one or more of the plurality of pregnancy-related state-associated genomic loci to generate the datasets indicative of a quantitative measure (e.g., indicative of a presence, absence, or relative amount) of sequences at each of the plurality of pregnancy-related state-associated genomic loci in the cell-free biological sample. For example, quantification of array hybridization or polymerase chain reaction (PCR) corresponding to the plurality of pregnancy-related state-associated genomic loci may generate the datasets indicative of a quantitative measure (e.g., indicative of a presence, absence, or relative amount) of sequences at each of the plurality of pregnancy-related state-associated genomic loci in the cell-free biological sample. Assay readouts may comprise quantitative PCR (qPCR) values, digital PCR (dPCR) values, digital droplet PCR (ddPCR) values, fluorescence values, etc., or normalized values thereof.

A kit may comprise a metabolomics assay for identifying a quantitative measure (e.g., indicative of a presence, absence, or relative amount) of each of a plurality of pregnancy-related state-associated metabolites in a cell-free biological sample of the subject. A quantitative measure (e.g., indicative of a presence, absence, or relative amount) of pregnancy-related state-associated metabolites in the cell-free biological sample may be indicative of one or more pregnancy-related states. The metabolites in the cell-free biological sample may be produced (e.g., as an end product or a byproduct) as a result of one or more metabolic pathways corresponding to pregnancy-related state-associated genes. A kit may comprise instructions for isolating or extracting the metabolites from the cell-free biological sample and/or for using the metabolomics assay to generate datasets indicative of the quantitative measure (e.g., indicative of a presence, absence, or relative amount) of each of a plurality of pregnancy-related state-associated metabolites in the cell-free biological sample of the subject.

### Trained Algorithms

After using one or more assays to process one or more cell-free biological samples derived from the subject to generate one or more datasets indicative of the pregnancy-related state or pregnancy-related complication, a trained algorithm may be used to process one or more of the datasets (e.g., at each of a plurality of pregnancy-related state-associated genomic loci) to determine the pregnancy-related state. For example, the trained algorithm may be used to determine quantitative measures of sequences at each of the plurality of pregnancy-related state-associated genomic loci in the cell-free biological samples. The trained algorithm may be configured to identify the pregnancy-related state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a differential expression algorithm. The differential expression algorithm may comprise a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, or a combination thereof. The trained algorithm may comprise an unsupervised machine learning algorithm.

The trained algorithm may be configured to accept a plurality of input variables and to produce one or more output values based on the plurality of input variables. The plurality of input variables may comprise one or more datasets indicative of a pregnancy-related state. For example, an input variable may comprise a number of sequences corresponding to or aligning to each of the plurality of pregnancy-related state-associated genomic loci. The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the cell-free biological sample by the classifier. The trained algorithm may comprise a binary classifier, such that each of the one or more output values comprises one of two values (e.g., {0, 1}, {positive, negative}, or {high-risk, low-risk}) indicating a classification of the cell-free biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the cell-free biological sample by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's pregnancy-related state, and may comprise, for example, a therapeutic intervention, a duration of the therapeutic intervention, and/or a dosage of the therapeutic intervention suitable to treat a pregnancy-related condition. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof. For example, such descriptive labels may provide a prognosis of the pregnancy-related state of the subject. As another example, such descriptive labels may provide a relative assessment of the pregnancy-related state (e.g., an estimated gestational age in number of days, weeks, or months) of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the pregnancy-related state of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a pregnancy-related state (e.g., pregnancy-related complication). For example, a binary classification of samples may assign an output value of “negative” or 0 if the sample indicates that the subject has less than a 50% probability of having a pregnancy-related state (e.g., pregnancy-related complication). In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a pregnancy-related state (e.g., pregnancy-related complication) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having a pregnancy-related state (e.g., pregnancy-related complication) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a pregnancy-related state (e.g., pregnancy-related complication) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a pregnancy-related state (e.g., pregnancy-related complication) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a cell-free biological sample from a subject, associated datasets obtained by assaying the cell-free biological sample (as described elsewhere herein), and one or more known output values corresponding to the cell-free biological sample (e.g., a clinical diagnosis, prognosis, absence, or treatment efficacy of a pregnancy-related state of the subject). Independent training samples may comprise cell-free biological samples and associated datasets and outputs obtained or derived from a plurality of different subjects. Independent training samples may comprise cell-free biological samples and associated datasets and outputs obtained at a plurality of different time points from the same subject (e.g., on a regular basis such as weekly, biweekly, or monthly). Independent training samples may be associated with presence of the pregnancy-related state (e.g., training samples comprising cell-free biological samples and associated datasets and outputs obtained or derived from a plurality of subjects known to have the pregnancy-related state). Independent training samples may be associated with absence of the pregnancy-related state (e.g., training samples comprising cell-free biological samples and associated datasets and outputs obtained or derived from a plurality of subjects who are known to not have a previous diagnosis of the pregnancy-related state or who have received a negative test result for the pregnancy-related state).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise cell-free biological samples associated with presence of the pregnancy-related state and/or cell-free biological samples associated with absence of the pregnancy-related state. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the pregnancy-related state. In some embodiments, the cell-free biological sample is independent of samples used to train the trained algorithm.

The trained algorithm may be trained with a first number of independent training samples associated with presence of the pregnancy-related state and a second number of independent training samples associated with absence of the pregnancy-related state. The first number of independent training samples associated with presence of the pregnancy-related state may be no more than the second number of independent training samples associated with absence of the pregnancy-related state. The first number of independent training samples associated with presence of the pregnancy-related state may be equal to the second number of independent training samples associated with absence of the pregnancy-related state. The first number of independent training samples associated with presence of the pregnancy-related state may be greater than the second number of independent training samples associated with absence of the pregnancy-related state.

The trained algorithm may be configured to identify the pregnancy-related state at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the pregnancy-related state by the trained algorithm may be calculated as the percentage of independent test samples (e.g., subjects known to have the pregnancy-related state or subjects with negative clinical test results for the pregnancy-related state) that are correctly identified or classified as having or not having the pregnancy-related state.

The trained algorithm may be configured to identify the pregnancy-related state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the pregnancy-related state using the trained algorithm may be calculated as the percentage of cell-free biological samples identified or classified as having the pregnancy-related state that correspond to subjects that truly have the pregnancy-related state.

The trained algorithm may be configured to identify the pregnancy-related state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the pregnancy-related state using the trained algorithm may be calculated as the percentage of cell-free biological samples identified or classified as not having the pregnancy-related state that correspond to subjects that truly do not have the pregnancy-related state.

The trained algorithm may be configured to identify the pregnancy-related state with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the pregnancy-related state using the trained algorithm may be calculated as the percentage of independent test samples associated with presence of the pregnancy-related state (e.g., subjects known to have the pregnancy-related state) that are correctly identified or classified as having the pregnancy-related state.

The trained algorithm may be configured to identify the pregnancy-related state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the pregnancy-related state using the trained algorithm may be calculated as the percentage of independent test samples associated with absence of the pregnancy-related state (e.g., subjects with negative clinical test results for the pregnancy-related state) that are correctly identified or classified as not having the pregnancy-related state.

The trained algorithm may be configured to identify the pregnancy-related state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying cell-free biological samples as having or not having the pregnancy-related state.

The trained algorithm may be adjusted or tuned to improve one or more of the performance, accuracy, PPV, NPV, clinical sensitivity, clinical specificity, or AUC of identifying the pregnancy-related state. The trained algorithm may be adjusted or tuned by adjusting parameters of the trained algorithm (e.g., a set of cutoff values used to classify a cell-free biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of pregnancy-related state-associated genomic loci may be identified as most influential or most important to be included for making high-quality classifications or identifications of pregnancy-related states (or sub-types of pregnancy-related states). The plurality of pregnancy-related state-associated genomic loci or a subset thereof may be ranked based on classification metrics indicative of each genomic locus's influence or importance toward making high-quality classifications or identifications of pregnancy-related states (or sub-types of pregnancy-related states). Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Identifying or Monitoring a Pregnancy-Related State

After using a trained algorithm to process the dataset, the pregnancy-related state or pregnancy-related complication may be identified or monitored in the subject. The identification may be based at least in part on quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites.

The pregnancy-related state may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the pregnancy-related state by the trained algorithm may be calculated as the percentage of independent test samples (e.g., subjects known to have the pregnancy-related state or subjects with negative clinical test results for the pregnancy-related state) that are correctly identified or classified as having or not having the pregnancy-related state.

The pregnancy-related state may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the pregnancy-related state using the trained algorithm may be calculated as the percentage of cell-free biological samples identified or classified as having the pregnancy-related state that correspond to subjects that truly have the pregnancy-related state.

The pregnancy-related state may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the pregnancy-related state using the trained algorithm may be calculated as the percentage of cell-free biological samples identified or classified as not having the pregnancy-related state that correspond to subjects that truly do not have the pregnancy-related state.

The pregnancy-related state may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the pregnancy-related state using the trained algorithm may be calculated as the percentage of independent test samples associated with presence of the pregnancy-related state (e.g., subjects known to have the pregnancy-related state) that are correctly identified or classified as having the pregnancy-related state.

The pregnancy-related state may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the pregnancy-related state using the trained algorithm may be calculated as the percentage of independent test samples associated with absence of the pregnancy-related state (e.g., subjects with negative clinical test results for the pregnancy-related state) that are correctly identified or classified as not having the pregnancy-related state.

In an aspect, the present disclosure provides a method for determining that a subject is at risk of pre-term birth, comprising assaying a cell-free biological sample derived from the subject to generate a dataset that is indicative of the pre-term birth risk at a specificity of at least 80%, and using a trained algorithm that is trained on samples independent of the cell-free biological sample to determine that the subject is at risk of pre-term birth at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

After the pregnancy-related state is identified in a subject, a sub-type of the pregnancy-related state (e.g., selected from among a plurality of sub-types of the pregnancy-related state) may further be identified. The sub-type of the pregnancy-related state may be determined based at least in part on the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites. For example, the subject may be identified as being at risk of a sub-type of pre-term birth (e.g., selected from among a plurality of sub-types of pre-term birth). After identifying the subject as being at risk of a sub-type of pre-term birth, a clinical intervention for the subject may be selected based at least in part on the sub-type of pre-term birth for which the subject is identified as being at risk. In some embodiments, the clinical intervention is selected from a plurality of clinical interventions (e.g., clinically indicated for different sub-types of pre-term birth).

In some embodiments, the trained algorithm may determine that the subject is at risk of pre-term birth of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm may determine that the subject is at risk of pre-term birth at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

Upon identifying the subject as having the pregnancy-related state, the subject may be optionally provided with a therapeutic intervention (e.g., prescribing an appropriate course of treatment to treat the pregnancy-related state of the subject). The therapeutic intervention may comprise a prescription of an effective dose of a drug, a further testing or evaluation of the pregnancy-related state, a further monitoring of the pregnancy-related state, an induction or inhibition of labor, or a combination thereof. If the subject is currently being treated for the pregnancy-related state with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

The quantitative measures of sequence reads of the dataset at the panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites may be assessed over a duration of time to monitor a patient (e.g., subject who has pregnancy-related state or who is being treated for pregnancy-related state). In such cases, the quantitative measures of the dataset of the patient may change during the course of treatment. For example, the quantitative measures of the dataset of a patient with decreasing risk of the pregnancy-related state due to an effective treatment may shift toward the profile or distribution of a healthy subject (e.g., a subject without a pregnancy-related complication). Conversely, for example, the quantitative measures of the dataset of a patient with increasing risk of the pregnancy-related state due to an ineffective treatment may shift toward the profile or distribution of a subject with higher risk of the pregnancy-related state or a more advanced pregnancy-related state.

The pregnancy-related state of the subject may be monitored by monitoring a course of treatment for treating the pregnancy-related state of the subject. The monitoring may comprise assessing the pregnancy-related state of the subject at two or more time points. The assessing may be based at least on the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined at each of the two or more time points.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of one or more clinical indications, such as (i) a diagnosis of the pregnancy-related state of the subject, (ii) a prognosis of the pregnancy-related state of the subject, (iii) an increased risk of the pregnancy-related state of the subject, (iv) a decreased risk of the pregnancy-related state of the subject, (v) an efficacy of the course of treatment for treating the pregnancy-related state of the subject, and (vi) a non-efficacy of the course of treatment for treating the pregnancy-related state of the subject.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a diagnosis of the pregnancy-related state of the subject. For example, if the pregnancy-related state was not detected in the subject at an earlier time point but was detected in the subject at a later time point, then the difference is indicative of a diagnosis of the pregnancy-related state of the subject. A clinical action or decision may be made based on this indication of diagnosis of the pregnancy-related state of the subject, such as, for example, prescribing a new therapeutic intervention for the subject. The clinical action or decision may comprise recommending the subject for a secondary clinical test to confirm the diagnosis of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a prognosis of the pregnancy-related state of the subject.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of the subject having an increased risk of the pregnancy-related state. For example, if the pregnancy-related state was detected in the subject both at an earlier time point and at a later time point, and if the difference is a negative difference (e.g., the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites increased from the earlier time point to the later time point), then the difference may be indicative of the subject having an increased risk of the pregnancy-related state. A clinical action or decision may be made based on this indication of the increased risk of the pregnancy-related state, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of the subject having a decreased risk of the pregnancy-related state. For example, if the pregnancy-related state was detected in the subject both at an earlier time point and at a later time point, and if the difference is a positive difference (e.g., the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites decreased from the earlier time point to the later time point), then the difference may be indicative of the subject having a decreased risk of the pregnancy-related state. A clinical action or decision may be made based on this indication of the decreased risk of the pregnancy-related state (e.g., continuing or ending a current therapeutic intervention) for the subject. The clinical action or decision may comprise recommending the subject for a secondary clinical test to confirm the decreased risk of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of an efficacy of the course of treatment for treating the pregnancy-related state of the subject. For example, if the pregnancy-related state was detected in the subject at an earlier time point but was not detected in the subject at a later time point, then the difference may be indicative of an efficacy of the course of treatment for treating the pregnancy-related state of the subject. A clinical action or decision may be made based on this indication of the efficacy of the course of treatment for treating the pregnancy-related state of the subject, e.g., continuing or ending a current therapeutic intervention for the subject. The clinical action or decision may comprise recommending the subject for a secondary clinical test to confirm the efficacy of the course of treatment for treating the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a non-efficacy of the course of treatment for treating the pregnancy-related state of the subject. For example, if the pregnancy-related state was detected in the subject both at an earlier time point and at a later time point, and if the difference is a negative or zero difference (e.g., the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites increased or remained at a constant level from the earlier time point to the later time point), and if an efficacious treatment was indicated at an earlier time point, then the difference may be indicative of a non-efficacy of the course of treatment for treating the pregnancy-related state of the subject. A clinical action or decision may be made based on this indication of the non-efficacy of the course of treatment for treating the pregnancy-related state of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In another aspect, the present disclosure provides a computer-implemented method for predicting a risk of pre-term birth of a subject, comprising: (a) receiving clinical health data of the subject, wherein the clinical health data comprises a plurality of quantitative or categorical measures of the subject; (b) using a trained algorithm to process the clinical health data of the subject to determine a risk score indicative of the risk of pre-term birth of the subject; and (c) electronically outputting a report indicative of the risk score indicative of the risk of pre-term birth of the subject.

In some embodiments, for example, the clinical health data comprises one or more quantitative measures of the subject, such as age, weight, height, body mass index (BMI), blood pressure, heart rate, glucose levels, number of previous pregnancies, and number of previous births. As another example, the clinical health data can comprise one or more categorical measures, such as race, ethnicity, history of medication or other clinical treatment, history of tobacco use, history of alcohol consumption, daily activity or fitness level, genetic test results, blood test results, imaging results, and fetal screening results.

In some embodiments, the computer-implemented method for predicting a risk of pre-term birth of a subject is performed using a computer or mobile device application. For example, a subject can use a computer or mobile device application to input her own clinical health data, including quantitative and/or categorical measures. The computer or mobile device application can then use a trained algorithm to process the clinical health data to determine a risk score indicative of the risk of pre-term birth of the subject. The computer or mobile device application can then display a report indicative of the risk score indicative of the risk of pre-term birth of the subject.

In some embodiments, the risk score indicative of the risk of pre-term birth of the subject can be refined by performing one or more subsequent clinical tests for the subject. For example, the subject can be referred by a physician for one or more subsequent clinical tests (e.g., an ultrasound imaging or a blood test) based on the initial risk score. Next, the computer or mobile device application may process results from the one or more subsequent clinical tests using a trained algorithm to determine an updated risk score indicative of the risk of pre-term birth of the subject.

In some embodiments, the risk score comprises a likelihood of the subject having a pre-term birth within a pre-determined duration of time. For example, the pre-determined duration of time may be about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks.

### Outputting a Report of the Pregnancy-Related State

After the pregnancy-related state is identified or an increased risk of the pregnancy-related state is monitored in the subject, a report may be electronically outputted that is indicative of (e.g., identifies or provides an indication of) the pregnancy-related state of the subject. The subject may not display a pregnancy-related state (e.g., is asymptomatic of the pregnancy-related state such as a pregnancy-related complication). The report may be presented on a graphical user interface (GUI) of an electronic device of a user. The user may be the subject, a caretaker, a physician, a nurse, or another health care worker.

The report may include one or more clinical indications such as (i) a diagnosis of the pregnancy-related state of the subject, (ii) a prognosis of the pregnancy-related state of the subject, (iii) an increased risk of the pregnancy-related state of the subject, (iv) a decreased risk of the pregnancy-related state of the subject, (v) an efficacy of the course of treatment for treating the pregnancy-related state of the subject, and (vi) a non-efficacy of the course of treatment for treating the pregnancy-related state of the subject. The report may include one or more clinical actions or decisions made based on these one or more clinical indications. Such clinical actions or decisions may be directed to therapeutic interventions, induction or inhibition of labor, or further clinical assessment or testing of the pregnancy-related state of the subject.

For example, a clinical indication of a diagnosis of the pregnancy-related state of the subject may be accompanied with a clinical action of prescribing a new therapeutic intervention for the subject. As another example, a clinical indication of an increased risk of the pregnancy-related state of the subject may be accompanied with a clinical action of prescribing a new therapeutic intervention or switching therapeutic interventions (e.g., ending a current treatment and prescribing a new treatment) for the subject. As another example, a clinical indication of a decreased risk of the pregnancy-related state of the subject may be accompanied with a clinical action of continuing or ending a current therapeutic intervention for the subject. As another example, a clinical indication of an efficacy of the course of treatment for treating the pregnancy-related state of the subject may be accompanied with a clinical action of continuing or ending a current therapeutic intervention for the subject. As another example, a clinical indication of a non-efficacy of the course of treatment for treating the pregnancy-related state of the subject may be accompanied with a clinical action of ending a current therapeutic intervention and/or switching to (e.g., prescribing) a different new therapeutic intervention for the subject.

### Computer Systems

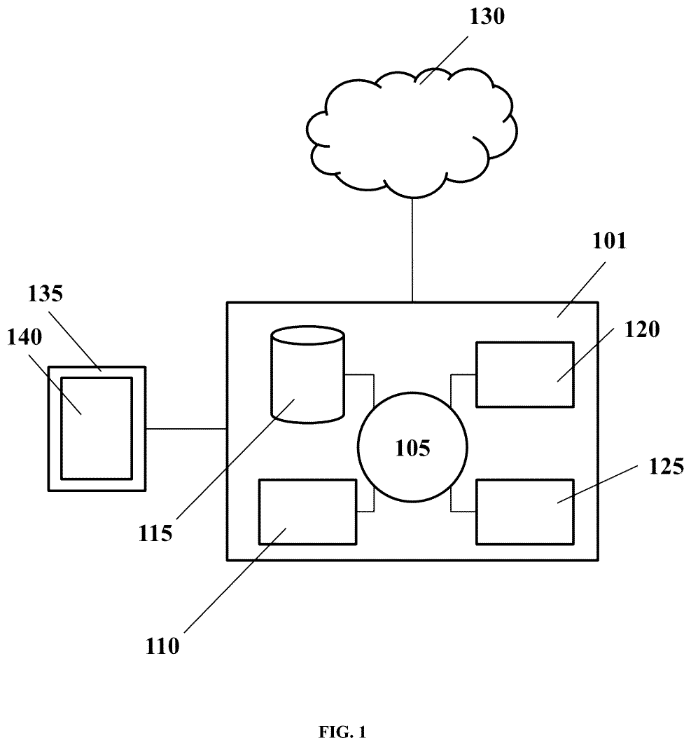

The present disclosure provides computer systems that are programmed to implement methods of the disclosure. FIG. 1 shows a computer system 101 that is programmed or otherwise configured to, for example, (i) train and test a trained algorithm, (ii) use the trained algorithm to process data to determine a pregnancy-related state of a subject, (iii) determine a quantitative measure indicative of a pregnancy-related state of a subject, (iv) identify or monitor the pregnancy-related state of the subject, and (v) electronically output a report that indicative of the pregnancy-related state of the subject.

The computer system 101 can regulate various aspects of analysis, calculation, and generation of the present disclosure, such as, for example, (i) training and testing a trained algorithm, (ii) using the trained algorithm to process data to determine a pregnancy-related state of a subject, (iii) determining a quantitative measure indicative of a pregnancy-related state of a subject, (iv) identifying or monitoring the pregnancy-related state of the subject, and (v) electronically outputting a report that indicative of the pregnancy-related state of the subject. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet.

The network 130 in some cases is a telecommunication and/or data network. The network 130 can include one or more computer servers, which can enable distributed computing, such as cloud computing. For example, one or more computer servers may enable cloud computing over the network 130 (“the cloud”) to perform various aspects of analysis, calculation, and generation of the present disclosure, such as, for example, (i) training and testing a trained algorithm, (ii) using the trained algorithm to process data to determine a pregnancy-related state of a subject, (iii) determining a quantitative measure indicative of a pregnancy-related state of a subject, (iv) identifying or monitoring the pregnancy-related state of the subject, and (v) electronically outputting a report that indicative of the pregnancy-related state of the subject. Such cloud computing may be provided by cloud computing platforms such as, for example, Amazon Web Services (AWS), Microsoft Azure, Google Cloud Platform, and IBM cloud. The network 130, in some cases with the aid of the computer system 101, can implement a peer-to-peer network, which may enable devices coupled to the computer system 101 to behave as a client or a server.

The CPU 105 may comprise one or more computer processors and/or one or more graphics processing units (GPUs). The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, (i) a visual display indicative of training and testing of a trained algorithm, (ii) a visual display of data indicative of a pregnancy-related state of a subject, (iii) a quantitative measure of a pregnancy-related state of a subject, (iv) an identification of a subject as having a pregnancy-related state, or (v) an electronic report indicative of the pregnancy-related state of the subject. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, (i) train and test a trained algorithm, (ii) use the trained algorithm to process data to determine a pregnancy-related state of a subject, (iii) determine a quantitative measure indicative of a pregnancy-related state of a subject, (iv) identify or monitor the pregnancy-related state of the subject, and (v) electronically output a report that indicative of the pregnancy-related state of the subject.

### EXAMPLES

### Example 1: Early Prediction of Spontaneous Preterm Birth and Late Miscarriages in a High-Risk Population Using Molecular Sub-Typing with cfRNA Profiling

Using systems and methods of the present disclosure, early molecular markers of preterm birth (PTB) and stillbirth were identified in maternal blood samples from a population at increased risk of PTB due to clinical history.

The study design was performed as follows. Blood samples from 229 women were collected between weeks 12-24 of gestation (IQR 18.9-20.9) and before onset of labor. Samples were collected across 4 independent sites in the UK, and 51% of the cohort were considered to be at high-risk, defined by at least one of: prior sPTB (16-36), previous cervical surgery, or a cervical length of less than 25 mm. 80% (n=183) delivered at term and 20% (n=46) had a sPTB (GA of less than 35 weeks). 30% of the sPTB delivered at a GA of less than 25 weeks. 80% (n=183) delivered at term and 20% (n=46) had a sPTB (GA of less than 35 weeks). 30% of the sPTB delivered at a GA of less than 25 weeks. All samples were processed using a unified experimental and computational NGS pipeline for cell-free (cfRNA) sequencing. Twin pregnancies and cases of preeclampsia were excluded.

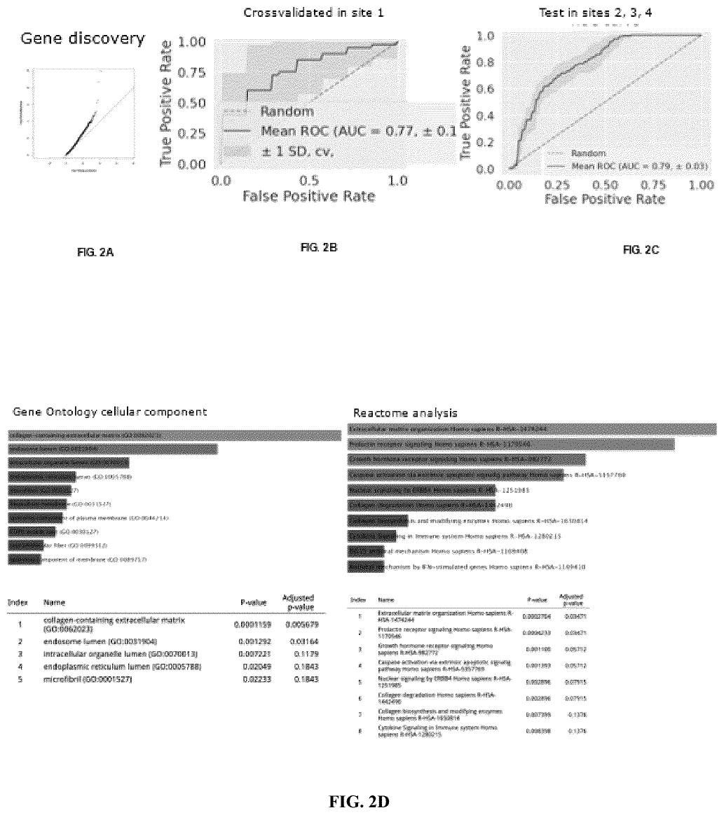

cfRNA profiling was performed of plasma obtained from all 229 maternal blood samples. 41 genes (COL3A1, HSD17B1, GPC4, CDR1-AS, COL5A1, COL6A1, COL1A1, EFHDI, LENG8, DCN, CYB5R2, ELN, ANTXR1, CH507-24F1.2, EIF4A1, ABI3BP, LAPTM5, SLC38A10, CCDC80, CIS, VPS45, COLIA2, C7, FN1, COL14A1, ARPC4-TTLL3, LINC01002, PSG4, TMTC2, STAG3L5P, AMT, TAPI, CSH1, MMP2, PLXNA3, LGMNP1, LUM, MYO18B, DAPK2, GCM1L, GALS14, FNDC10, FBN2, CAPN13, TNFRSF25, MYH11, POGLUT1, GH2, DNAH1, DES, NUP210) were identified as being associated with an increased risk of sPTB (FDR<0.1). Based on these transcripts, a logistic regression classifier model was developed to predict PTB, obtaining an AUC=0.72, as shown in FIGS. 2A-2C. The model was validated through leave-one-out cross-validation (LOOCV). Additional insight in the pathophysiology of PTB are presented in FIG. 2D. Pathway analysis for the transcripts associated with sPTB revealed an enrichment of genes related to collagen-containing and extracellular matrix in those individuals who ultimately had a sPTB at a GA of less than 35 weeks.

Additional analysis for extremely late miscarriage/early pre-term birth (GA of less than 25 weeks at delivery) was performed on 14 cases and 166 at term controls. A logistic regression classifier was developed based on 11 genes (AC011043.1, IGFBP2, SH3GL3, AMT, GTF2IP4, GYPB, PAPPA, CH17-472G23.2, OMA1, ACADSB, ACER3) to predict risk of extremely late miscarriage/early pre-term birth with an enhanced performance of AUC=0.76 in LOOCV. While enrichment for were associated with sPTB at a GA of less than 25 weeks.

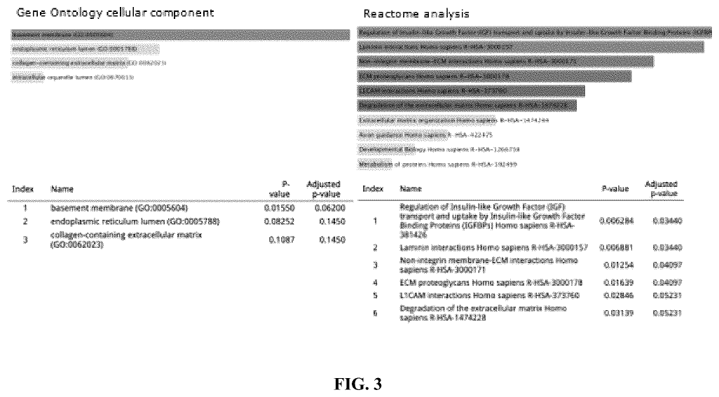

Pathway analysis of the 6-transcript set revealed an enrichment of a set of genes related to basement membrane and endoplasmic reticulum lumen, and genes in insulin-like growth factor transport and uptake and amino acid metabolism pathways in samples from maternal subjects that deliver before a GA of 25 weeks, providing additional insight in the pathophysiology of extremely late miscarriage/early pre-term birth as presented in FIG. 3.

The analysis of cfRNA in maternal plasma provided a noninvasive window to maternal-fetal health in pregnancies at increased risk of pregnancy complications. Our data showed that elevated expression of a subset of transcripts potentially involved in at least two molecular sub-type of preterm birth and two different underlining mechanisms. In a first molecular sub-type, a collagen-containing extracellular matrix pathway may be associated with cervical remodeling associated with shorter cervix, and cervix insufficiency may form a basis for underling biology for high risk PTB delivery. In a second molecular sub-type, for endoplasmic reticulum lumen pathway, there may be an association of endoplasmic reticulum stress induced by oxidative stress in decidual cells with a possible mechanism of early pregnancy loss. Further, a basement membrane pathway may indicate the premature placental membrane separation from uterus during miscarriage.

Based on these results of the molecular sub-typing for PTB, specific treatments may be selected and administered to maternal subjects based on a particular molecular subtype, to modulate the outcome of pre-term birth. For example, collagen modulating therapeutics or cervical cerclage can be applied to stabilize the cervix. The late miscarriage cases may be prevented by administering therapeutics to reduce oxidative stress and/or modulating expression levels of proteins related to endoplasmic reticulum stress.

### Example 2: Early Prediction of Preeclampsia Using Molecular Sub-Typing with cfRNA Profiling

Using systems and methods of the present disclosure, early molecular markers of preeclampsia (PE) are identified in maternal blood samples from a population at increased risk of preeclampsia (PE) due to clinical history.

Further, a cohort of subjects includes a set of control subjects with delivery after 37 weeks of gestational age. Some control subjects are classified as healthy controls, and some control subjects have a history of chronic hypertension without preeclampsia. A set of case subjects are diagnosed with preeclampsia with delivery before 37 weeks of gestational age. A set of case subjects are diagnosed with de novo preeclampsia, and a subset of case subjects have preeclampsia with a history of chronic hypertension.

Differential expression analysis of the cohort data set is performed as follows. Biomarker discovery is performed to identify early diagnostic markers of preeclampsia using cell-free RNA. In order to estimate the effect of chronic hypertension, two separate differential expression analyses are performed to estimate the effect of chronic hypertension. A first analysis is performed on a set of preeclampsia cases and a set of healthy controls; further, a second analysis is performed, in which a set of control subjects with chronic hypertension is added, thereby totaling a larger number of control subjects.

A set of top differentially expressed genes for PE in the cohort is identified for both comparisons including chronic hypertension and excluding chronic hypertension. The top genes from both analyses are observed to overlap, which is indicative of a signal associated with preeclampsia, and not chronic hypertension.

Additional analysis of highly significant genes associated with higher risk of PE indicates at least two separate pathways with different underlying biology. In a first molecular sub-type, the enrichment of low expressed placenta-specific genes like PAPA2 and FABP1 indicates significant changes in early placentation, which may be associated with preeclampsia. Also, a high dose of aspirin administered at early pregnancy before 13 weeks may reduce the risk of extremely early onset of PE (<32 weeks) but may not reduce the PE developed later. In a second molecular sub-type, the pathways associated with keratinocyte endothelium may be associated with vascular inflammation, endothelial dysfunction, and arterial hypertension. Moreover, the skin holds a complex capillary counter current system which controls body temperature, skin perfusion, and apparently systemic blood pressure. Therefore, the case of once of preeclampsia can be associated with underlying biology of mother's skin, capillary, and or arterial dysfunction.

Based on these results of molecular sub-typing for PE, specific treatments may be selected and administered to maternal subjects to modulate the outcome or risk of developing preeclampsia. For example, subjects with a molecular sub-type of placentation may be treated with compounds similar to aspirin, which is associated with regulation of cyclooxygenase pathways or pathways responsible for modulating the vasodilatory mediators and inhibiting vascular remodeling, platelet aggregation, and platelet adhesion. As another example, subjects with a molecular sub-type of PE associated with keratinocyte endothelium pathways may be treated with blood pressure management compounds or compounds targeted to a mechanism of proton pump inhibitors (PPI).

Blocking PPI may lead to decreased sFlt-1 and soluble endoglin (sENG) secretion and endothelial dysfunction, dilation of blood vessels, decreased BP, and antioxidant and anti-inflammatory properties. Use of Esomeprazole, another proton pump inhibitor that is also used for gastric reflux, may be evaluated in phase II clinical studies to treat early onset PE (PIE Trail) in maternal subjects.

### Example 3: Early Prediction of Gestational Diabetes Mellitus (GDM) Using Molecular Sub-Typing with cfRNA Profiling

Using systems and methods of the present disclosure, early molecular markers of diabetes mellitus (GDM) are identified in maternal blood samples from a population at increased risk of GDM due to clinical history.

Further, a cohort of subjects includes a set of control subjects. Some control subjects are classified as healthy controls with negative Oral Glucose Tolerance Test (OGTT) test. A first set of case subjects are diagnosed with gestational GDM based on OGTT test, a second set of case subjects are diagnosed with chronic Type 2 diabetes, and a third set of case subjects have impaired glucose status.

Differential expression analysis of the cohort data set is performed as follows. Biomarker discovery is performed to identify early diagnostic markers of GDM using cell-free RNA. In order to estimate the effect of chronic Type 2 diabetes, two separate differential expression analyses are performed to estimate the effect. A first analysis was performed on a set of gestational GDM cases and a set of healthy controls; further, a second analysis is performed, in which a set of control subjects with chronic Type 2 diabetes are added, thereby totaling a larger number of control subjects.

A set of top differentially expressed genes for GDM in the cohort is identified for both comparisons including chronic Type 2 diabetes and excluding chronic Type 2 diabetes. The top genes from both analyses are observed to overlap, which is indicative of a signal associated with GDM, and not chronic Type 2 diabetes.

Additional analysis of highly significant genes associated with higher risk of GDM indicates at least three separate pathways with different underlying biology. In a first molecular sub-type, the enrichment of low expressed placenta-specific genes (PDK4, CSH1, and PLAC4) indicates significant changes as placenta deterioration, placenta insufficiency, placenta failure, placenta dysfunction, premature ageing, calcification and impaired placenta function with gestational diabetes. In a second molecular sub-type, one of genes TBCEL, tubulin-specific chaperone cofactor E-like, may be associated with mediated hyperglycemic memory, which may be common for type 1 and type 2 diabetes. In a third molecular sub-type, FBXO7 gene is involved in adaptive immune system and antigen-specific immune response efficiently pathways. GDM is characterized not only by increased insulin resistance and glucose intolerance, but also by a state of low-grade systemic inflammation and dysregulation of the immune system which induces an imbalance between type 1 and 2 T-helper cells

Based on these results of molecular sub-typing for GDM, specific treatments may be selected and administered to maternal subjects to modulate the outcome or risk of developing GDM. For example, subjects with a molecular sub-type indicative of significant changes in placenta disfunction or deterioration may be treated with potential candidate drug targets-effectors to improve uteroplacental blood flow, anti-oxidants, heme oxygenase induction, inhibition of HIF, induction of cholesterol synthesis pathways, increasing insulin-like growth factor II availability. As another example, for subjects with second molecular sub-type associated with mediated hyperglycemic memory, an early aggressive treatment of this glucose imbalance may be administered to subjects with diabetes. Hyperglycemia may be accompanied by the formation of advanced glycation end products (AGEs). Another therapeutic approach may be to attempt to reduce AGE formation, receptor of AGE (RAGE) expression, and oxidative stress generation. Different drugs may be administered to block AGE formation, such as metformin and pioglitazone. ACE inhibitors and AT-1 blockers are compounds used to control blood pressure; however, they are also capable of reducing AGEs formation. Telmisartan downregulates RAGE mRNA levels and subsequently inhibits superoxide generation, whereas gliclazide may be useful in abolishing the “memory”. In addition, GLPI receptor agonists may be administered to decrease inflammation, postprandial hyperlipidemia, and coagulation, resulting in a beneficial effect on atherothrombosis. Aldose reductase inhibitors like Epalrestat may be administered to protect against diabetic peripheral neuropathy by alleviating oxidative stress and inhibiting polyol pathway. As another example, for subjects with a third molecular sub-type involved in adaptive immune system and antigen-specific pathways, pregnancy is a significant metabolic and immune challenge; further, GDM superimposes an enhanced degree of low-grade systemic inflammation and uncompensated insulin resistance, which may be further linked to a dysregulation of the underlying immune response, which may be common for Type 1 and Type 2. Immunomodulators may be administered to treat diabetes, such as: Azathioprine, Mycophenolate mofeti, Otelixizumab, Teplizumab (which may minimize cytokine release and prevent the progressive destruction of β-cells).

### Example 4: Predictive RNA Profiles for Early and Very Early Spontaneous Preterm Birth in a High-Risk Population and Pathway Analysis Using the Reactome Database

Using systems and methods of the present disclosure, early molecular markers of preterm birth (PTB) and very early spontaneous preterm birth (sPTB) were identified in maternal blood samples from a population to clinical history of high risk.

High-risk pregnancies were defined by at least one of: prior sPTB or late miscarriage (between 12 to 37 weeks of gestation), previous destructive cervical surgery, or incidental finding of a cervical length <25 mm on transvaginal ultrasound scan. Women with no risk factors for sPTB and otherwise well at the time of enrollment were recruited as low-risk controls from routine antenatal or ultrasonography clinics.

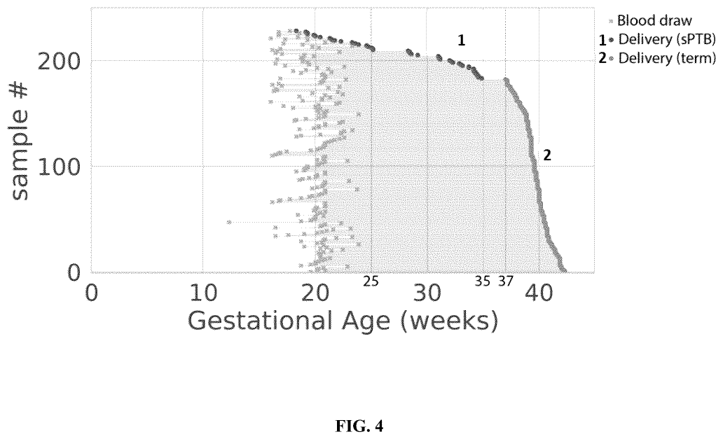

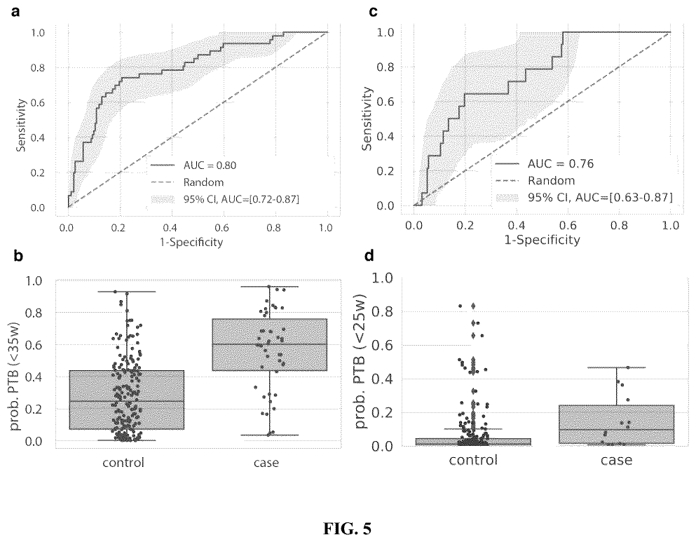

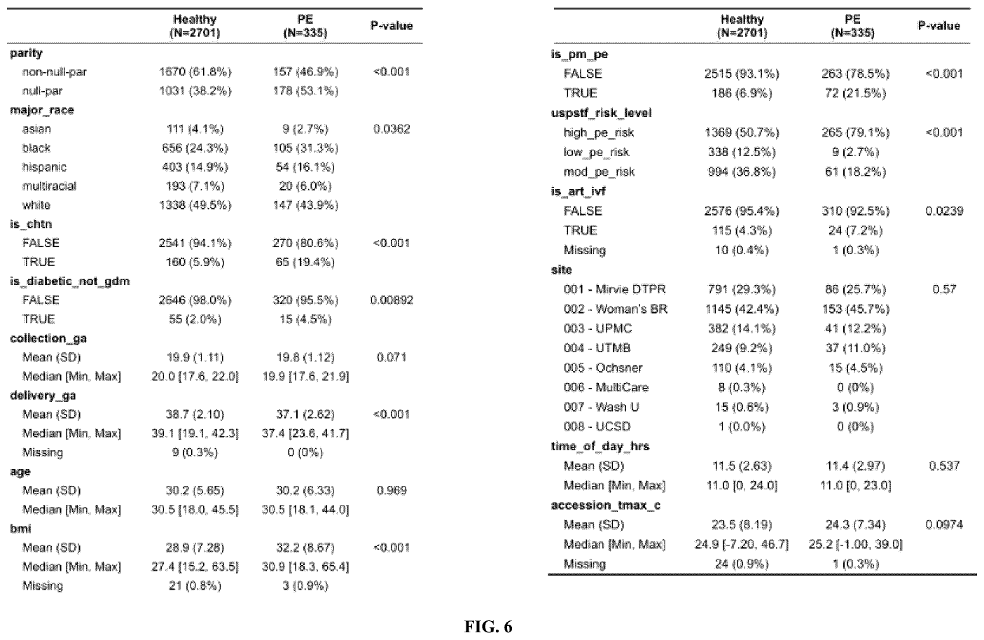

Blood samples were collected between 12 and 24 weeks of gestation (242 blood samples, one sample per pregnancy) from women with singleton pregnancies recruited from four tertiary antenatal clinics. For sPTB cases, samples were collected on average 9.4 weeks before delivery. Distribution of 242 collected samples with gestational age at blood sample collection, and gestational age at delivery are shown in FIG. 4. Out of 242 pregnancies, 194 delivered at term (≥37 0/7 GA), and 48 spontaneously delivered preterm before 35 weeks gestation (early preterm, <35 0/7). A subset of 16 of the pregnancies delivered before 25 weeks gestation (very early preterm, <25 0/7).

To identify candidate genes that can be predictive of risk of early sPTB (<35 0/7), differential expression analyses were performed between all early deliveries (<35 0/7) and controls (≥37 0/7). Results were validated using Leave-One-Out Cross-Validation (LOOCV), which resulted in a list of 25 differentially expressed genes listed in Table 1 that were used to build a logistic regression classifier to predict risk of preterm birth.

TABLE 1
Early sPTB differentially expressed genes identified by LOOCV
with corresponding identification frequency across the folds.

| Index | Gene | % Folds identified |
|---|---|---|
| 1 | COL14A1 | 100.0 |
| 2 | GCM1 | 100.0 |
| 3 | CH507-24F1.2 | 100.0 |
| 4 | GH2 | 100.0 |
| 5 | CYB5R2 | 100.0 |
| 6 | CAPN13 | 98.8 |
| 7 | STAG3L5P | 100.0 |
| 8 | GPC4 | 99.2 |
| 9 | LGALS14 | 100.0 |
| 10 | ELN | 98.3 |
| 11 | NPR2 | 100.0 |
| 12 | TNFRSF25 | 100.0 |
| 13 | HRH1 | 98.8 |
| 14 | ZNF404 | 100.0 |
| 15 | SIGLEC8 | 91.3 |
| 16 | MTHFD2P7 | 93.3 |
| 17 | PAGE4 | 50.4 |
| 18 | RGPD5 | 4.2 |
| 19 | ZNF812P | 6.7 |
| 20 | MTCO3P12 | 2.9 |
| 21 | AL773572.7 | 2.1 |
| 22 | LINC00969 | 0.4 |
| 23 | OLFML3 | 0.8 |
| 24 | FLG2 | 0.4 |
| 25 | AP000580.1 | 0.4 |

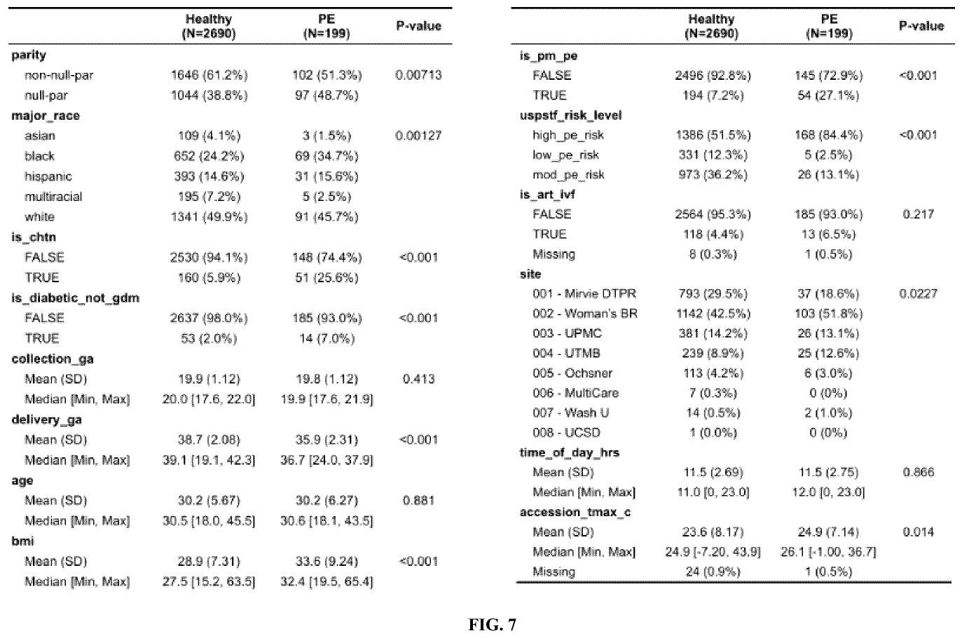

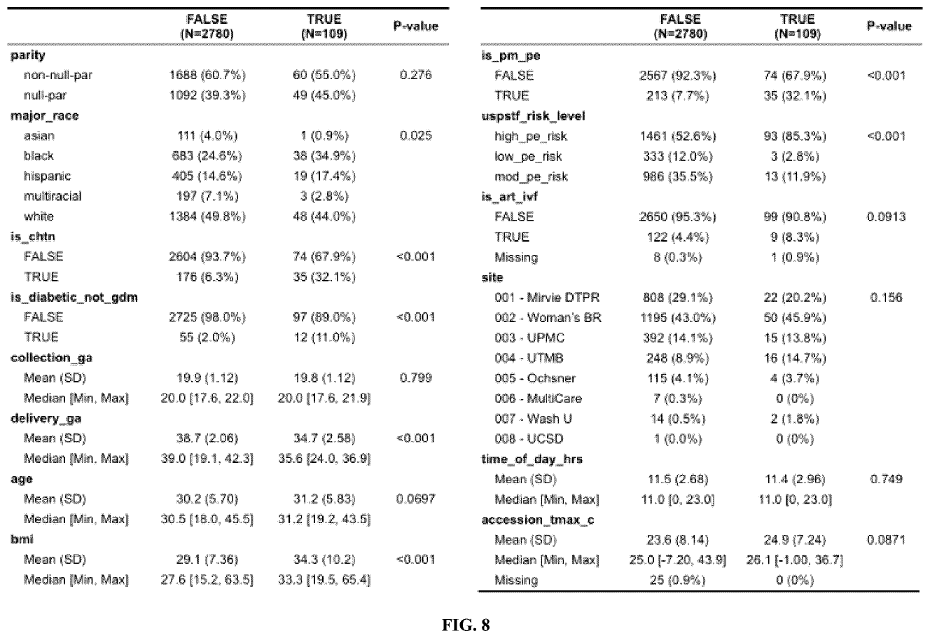

The model achieved a validated LOOCV performance area under the curve (AUC) of 0.80 (95% CI 0.72-0.87) with sensitivity=0.76 and specificity=0.72 (N=46 early sPTB cases and N=183 at-term controls) shown in FIG. 5A. The model also scored each sample with a risk probability of preterm delivery shown in FIG. 5B.

The same approach was used to identify molecular markers specific to very early sPTB (<25 weeks). Differential expression analyses were performed between 16 very early PTB cases and 226 controls, and the results were validated using LOOCV. A list of 65 differentially expressed genes was generated (Table 2), that were used to build a regularized logistic regression classifier to predict very early sPTB in cross-validation.

TABLE 2
Very early sPTB differentially expressed genes identified by LOOCV
with corresponding identification frequency across the folds.

| Index | Gene | % Folds Identified |
|---|---|---|
| 1 | AC011043.1 | 96.3 |
| 2 | SCN3A | 93.8 |
| 3 | SMAD5 | 88.0 |
| 4 | PLAC4 | 81.8 |
| 5 | TUBB2A | 74.4 |
| 6 | SEL1L3 | 74.0 |
| 7 | MCM6 | 69.0 |
| 8 | CUX2 | 68.6 |
| 9 | PPL | 60.7 |
| 10 | PRKG2 | 57.9 |
| 11 | CATSPERB | 55.8 |
| 12 | ACE | 54.1 |
| 13 | GTF2IP4 | 46.3 |
| 14 | KRT5 | 43.0 |
| 15 | AGPAT4 | 33.5 |
| 16 | ZCCHC7 | 33.1 |
| 17 | CYP19A1 | 28.9 |
| 18 | RGPD8 | 26.0 |
| 19 | TMEM70 | 26.0 |
| 20 | MTRNR2L1 | 26.0 |
| 21 | DDX11L10 | 21.5 |
| 22 | OLFM1 | 18.2 |
| 23 | C1orf21 | 9.5 |
| 24 | RPH3AL | 9.5 |
| 25 | LURAP1L | 7.0 |
| 26 | SPATA7 | 5.8 |
| 27 | CH17-472G23.2 | 4.5 |
| 28 | MXRA7 | 3.7 |
| 29 | PARD3B | 3.3 |
| 30 | UPK1A-AS1 | 3.3 |
| 31 | MT-ND6 | 2.9 |
| 32 | MKRN9P | 2.5 |
| 33 | FUT8 | 2.5 |
| 34 | ZNF528 | 2.5 |
| 35 | H3F3BP1 | 2.1 |
| 36 | FAM83D | 2.1 |
| 37 | AP003068.18 | 2.1 |
| 38 | CH17-431G21.1 | 1.7 |
| 39 | SH3GL3 | 1.7 |
| 40 | GSTM1 | 1.7 |
| 41 | CSH1 | 1.2 |
| 42 | GALNT12 | 1.2 |
| 43 | FCGR2A | 0.8 |
| 44 | EPS8L1 | 0.8 |
| 45 | RP11-514O12.4 | 0.8 |
| 46 | ZNF117 | 0.8 |
| 47 | PODXL | 0.8 |
| 48 | DHRSX | 0.8 |
| 49 | CD79A | 0.4 |
| 50 | CMA1 | 0.4 |
| 51 | XKR8 | 0.4 |
| 52 | FAM171A1 | 0.4 |
| 53 | DHCR7 | 0.4 |
| 54 | GPX1P1 | 0.4 |
| 55 | PHGDH | 0.4 |
| 56 | PAX8-AS1 | 0.4 |
| 57 | CNFN | 0.4 |
| 58 | PRPSAP1 | 0.4 |
| 59 | C5orf34 | 0.4 |
| 60 | LYSMD4 | 0.4 |
| 61 | IGFBP5 | 0.4 |
| 62 | TRBV20-1 | 0.4 |
| 63 | IGLC3 | 0.4 |
| 64 | KCNG1 | 0.4 |
| 65 | PPP2CB | 0.4 |

The model achieved a validated LOOCV performance of AUC-0.74 (95% CI 0.64-0.83). Several genes were found to be related genes involved in preeclampsia toxemia (PET). To reduce crosstalk in the cfRNA signature across the two complications, modeling was performed by excluding all PET samples and samples with low quality sequencing metrics, given the reduction to 14 cases of very early preterm (<25 0/7). The model exhibited improved LOOCV performance with AUC=0.76 (95% CI 0.63-0.87) [sensitivity=0.64, specificity=0.80] for 14 very early sPTB cases (<25 0/7) and 193 samples that delivered at or after 25 weeks (≥25 0/7), as shown in FIG. 5C. The model was based on a set of 39 differentially expressed genes listed in Table 3, from which a core set of three genes (AC011043.1, IGFBP2, and SH3GL3) was identified in >95% of cross-validation folds, and 13 genes overlapped with the differentially expressed genes discovered when training with the PET samples. The model probabilities showed a significant difference between cases and controls, although a longer tail of high sPTB probabilities was observed for a subset of control samples, as shown in FIG. 5D.

TABLE 3
Very early sPTB differentially expressed genes identified
by LOOCV with corresponding identification frequency
across the folds after exclusion of PET samples.

| Index | Gene | % Folds Identified |
|---|---|---|
| 1 | AC011043.1 | 100.0 |
| 2 | IGFBP2 | 99.5 |
| 3 | SH3GL3 | 95.7 |
| 4 | AMT | 89.4 |
| 5 | GTF2IP4 | 85.0 |
| 6 | GYPB | 81.6 |
| 7 | PAPPA | 56.5 |
| 8 | CH17-472G23.2 | 33.3 |
| 9 | OMA1 | 26.6 |
| 10 | ACADSB | 23.7 |
| 11 | ACER3 | 20.8 |
| 12 | MXRA7 | 9.2 |
| 13 | PCTP | 6.8 |
| 14 | TUBB2A | 6.8 |
| 15 | RNY4 | 6.3 |
| 16 | FAM171A1 | 6.3 |
| 17 | ILDR2 | 4.3 |
| 18 | NDST3 | 3.9 |
| 19 | MISP3 | 2.9 |
| 20 | GSTM2 | 2.9 |
| 21 | PHGDH | 2.4 |
| 22 | C1orf21 | 2.4 |
| 23 | TMEM140 | 1.9 |
| 24 | FAM111B | 1.9 |
| 25 | DDX11L10 | 1.4 |
| 26 | PPBP | 1.4 |
| 27 | MT-TV | 1.0 |
| 28 | MOB3C | 1.0 |
| 29 | RUNX1T1 | 1.0 |
| 30 | NEDD8-MDP1 | 1.0 |
| 31 | AMD1 | 1.0 |
| 32 | FAM83D | 1.0 |
| 33 | BMS1P10 | 0.5 |
| 34 | RAI14 | 0.5 |
| 35 | RNF144A | 0.5 |
| 36 | VPS37B | 0.5 |
| 37 | CATSPERB | 0.5 |
| 38 | FUT8 | 0.5 |
| 39 | TCEAL8 | 0.5 |

The analysis of biological pathways driving sPTB predictive genes was performed using the Reactome database and top two pathways for each preterm models listed in Table 4.

TABLE 4
Pathway analysis of sPTB differentially expressed genes. Pathway
analysis for genes discovered in the early sPTB predictor
(<35 0/7) and the very early sPTB predictor (<25 0/7).

| | | |
|---|---|---|
| Term | P-value | Adjusted P-value |

Early sPTB predictor (<35 0/7)

| | | |
|---|---|---|
| Extracellular matrix organization | 5.12E−03 | 0.098 |
| (R-HSA-1474244) | | |
| Degradation of the extracellular | 7.71E−03 | 0.098 |
| matrix (R-HSA-1474228) | | |

Very early sPTB predictor (<25 0/7)

| | | |
|---|---|---|
| Regulation of Insulin-like | 7.60E−04 | 0.043 |
| Growth Factor (IGF) | | |
| transport and uptake by | | |
| IGFBP (R-HSA-381426) | | |
| Metabolism of amino acids and | 4.01E−03 | 0.112 |
| derivatives (R-HSA-71291) | | |

The early preterm birth model (<35 0/7) was enriched for genes involved in extracellular matrix (ECM) degradation and remodeling. The data were in agreement with the observation that mediators of cell-to-cell adhesion such as undulin (COL14A1) and elastin (ELN) are among the top genes in the model. Early detection of ECM pathways from a blood draw may serve to identify individuals at risk for premature cervical remodeling. Such a screening test may be implemented at a similar window as ultrasound to measure cervical length in women at high-risk.

By contrast, a similar analysis performed on the genes obtained in the very early preterm birth model (<25 0/7 weeks) revealed that pathways related to insulin-like growth factor transport and amino acid metabolism pathways were observed to be differentially expressed in very early sPTB. Insulin binding proteins are highly expressed by the fetus and in the decidua basalis, and are key regulators of the bioavailability of IGF-1 and hence fetal growth. For instance, IGFBP1 is associated with intrauterine growth restriction and impaired placentation, and is raised in cord blood from extremely preterm infants. The detection of insulin-growth factor cfRNA, as a predominant signal in pregnancies that result in very early sPTB is both plausible and potentially informative in terms of downstream events.

### Example 5: Prospective Cohort Subjects for Preeclampsia Case Studies and cfRNA NGS Data Correction

Using systems and methods of the present disclosure, a prospective, observational study was performed of a cell-free RNA platform utilizing direct-to-participant recruitment via targeted social media from July 2020 to April 2022. The IRB-approved study was open to subjects of ages 18 to 45 with a singleton pregnancy in the United States. Participants signed informed consent, provided record release forms, completed a short questionnaire, and submitted blood samples through mobile phlebotomy scheduled via a web-based platform.

Participants submitted blood samples between 17 and 22 weeks of gestation age. Medical records were received for over 85% of participants. The cohort is geographically and ethnically diverse, representing 1,220 zip codes across 30 states. All samples were processed using a unified experimental and computational NGS pipeline for cell-free RNA (cfRNA) sequencing.

3,036 samples had complete medical records and passed cfRNA assay quality metrics. FIG. 6 shows the demographic and clinical data metrics for the preeclampsia observational studies, including 2,701 healthy participants and 335 participants diagnosed with preeclampsia.

FIG. 6 shows the distribution of demographic and clinical factors within this cohort associated with risk of developing preeclampsia, which were collected based on U.S. Preventive Services Task Force (USPSTF) guidelines and recommendations. Various preeclampsia risk factors were recorded for this cohort, including: parity; race; chronic hypertension (chtn); diabetic status excluding gestational diabetes (diabetic_not_gdm); mothers age; body mass index (bmi); prior preeclampsia diagnosis (pm_pe); U.S. Preventive Services Taskforce (USPSTF) risk level (www.uspreventiveservicestaskforce.org/uspstf/recommendation/preeclampsia-screening); and artificial in vitro fertilization (IVF).

To define the different subtypes of preeclampsia, different cutoffs for medically prescribed deliveries (delivery_GA) for preeclampsia-diagnosed patients were used. FIG. 7 shows the demographic of the same cohort of 2,889 samples for preeclampsia with delivery at less than 38 weeks, including 2,690 healthy participants and 199 participants who were diagnosed with preeclampsia and delivered before 38 weeks of gestational age.

FIG. 8 shows the demographic of the same cohort of 2,889 samples for preeclampsia with delivery at less than 37 weeks, including 2,780 healthy participants and 109 participants who were diagnosed with preeclampsia and delivered before 37 weeks of gestational age.

Cell-free RNA (cfRNA) level measures in plasma quantified by NGS techniques can be affected by systematic variation due to the technical processing of samples, which may compromise the accuracy of the measurement process and contribute to bias the estimate of the association under investigation. The quantification of the contribution of the systematic source of variation is challenging in datasets characterized by hundreds of thousands of features.

Several sources of systematic variations in the 3,036-sample cfRNA data set were identified, and several statistical correction methodologies were applied. These correction techniques included several methodologies: 1) residuals from multivariate linear regression were used to correct the data residuals, 2) a ComBat method was performed based on an empirical Bayes approach that can correct only for one covariate at the time; 3) and surrogate variables analysis (SVA) was developed to remove pre-identified sources of variability but also unknown sources of variability. The correction methodology using residuals from multivariate linear regression to correct the data for the effects of unwanted covariates demonstrated better correction on a complete dataset as compared to the other two techniques.

Various sources of variation were identified by analysis of the 3,036-sample cfDNA data set, and were successfully corrected by performing various methodologies. First, the technical variations attributed to NGS-specific methodology such as: depth of sequencing per sample; batch effects for individual process operations; or various raw materials were identified and corrected.

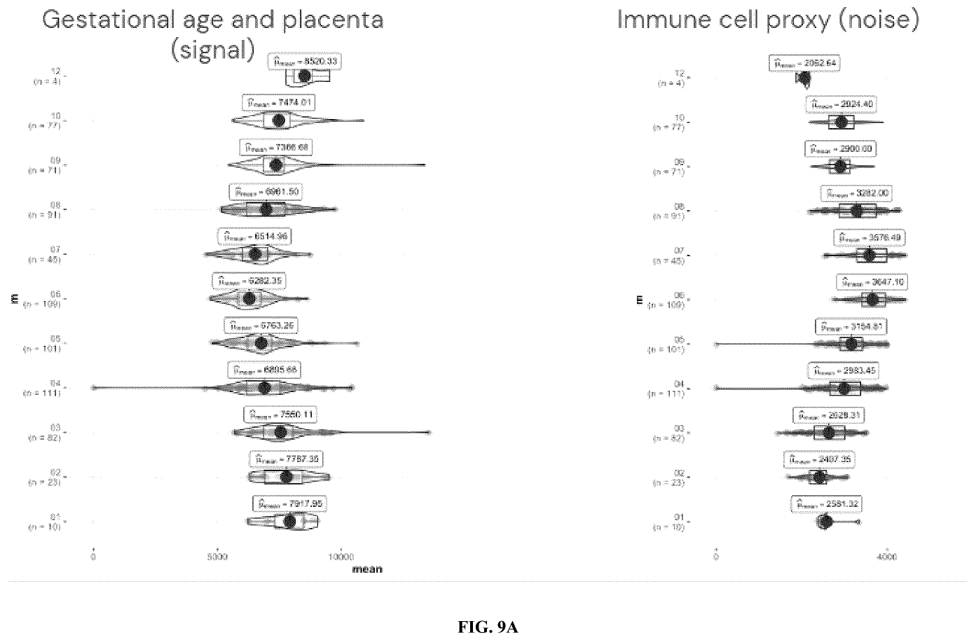

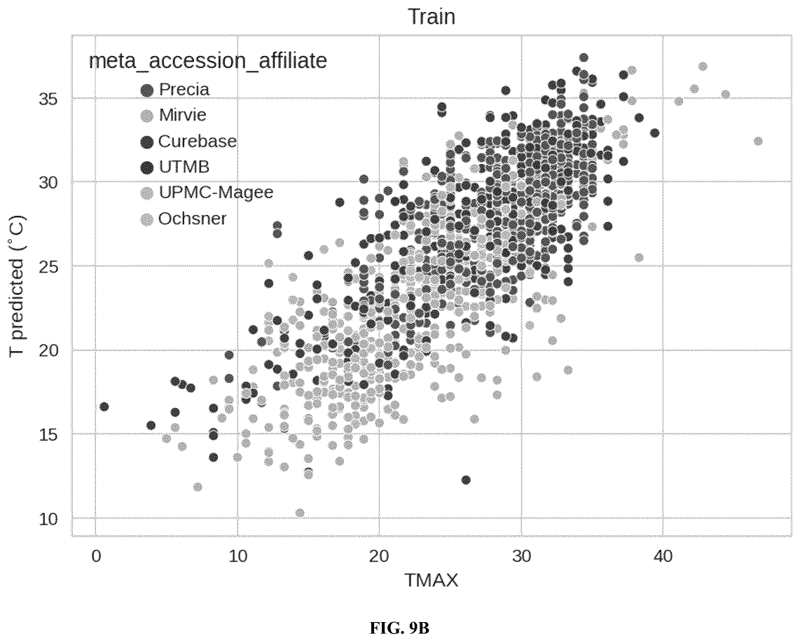

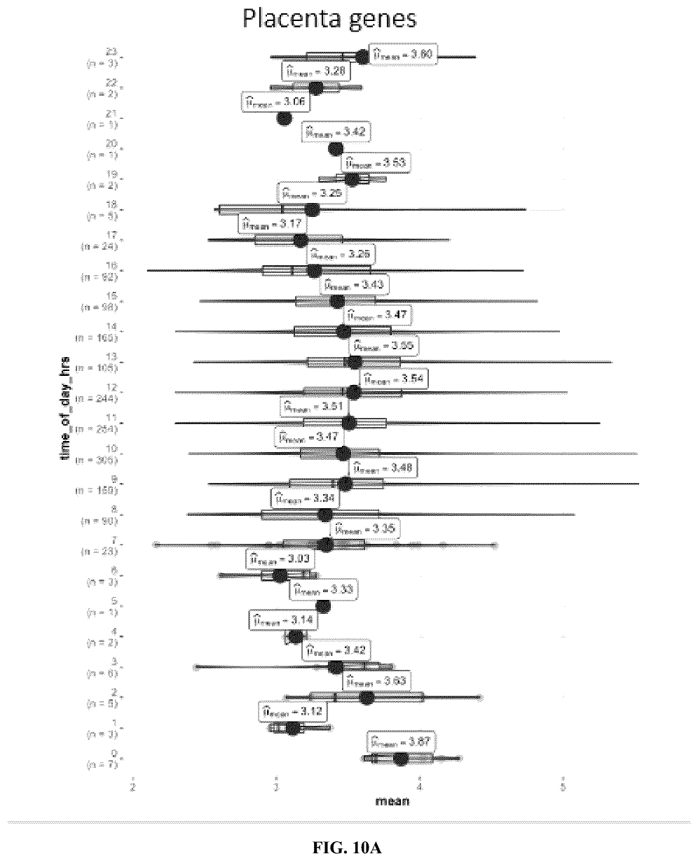

Further, two external sources of variation were identified related to time of blood collection, and corrected by multivariate linear regression to correct the data residuals. FIG. 9A shows an example of systematic variance in NGS data associated with seasonal changes for gestational- and placenta-associated genes moving in the opposite direction as the immune cellular genes. This set of genes was analyzed and determined to be highly correlated with outside temperature recorded at the weather station closest to the blood collection site at time of day of blood collection. FIG. 9B shows an example of high correlation between prediction of local outside temperature by gene modeling and actual temperatures recorded by weather station close to the blood collection site.

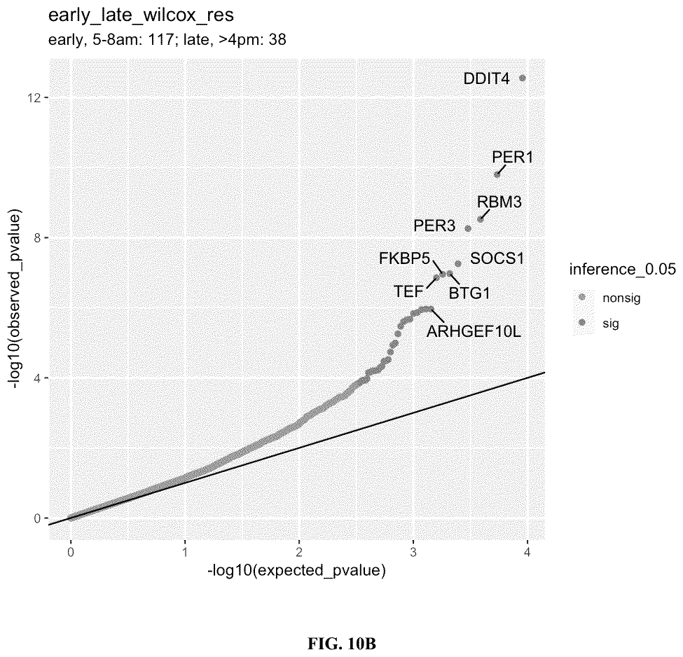

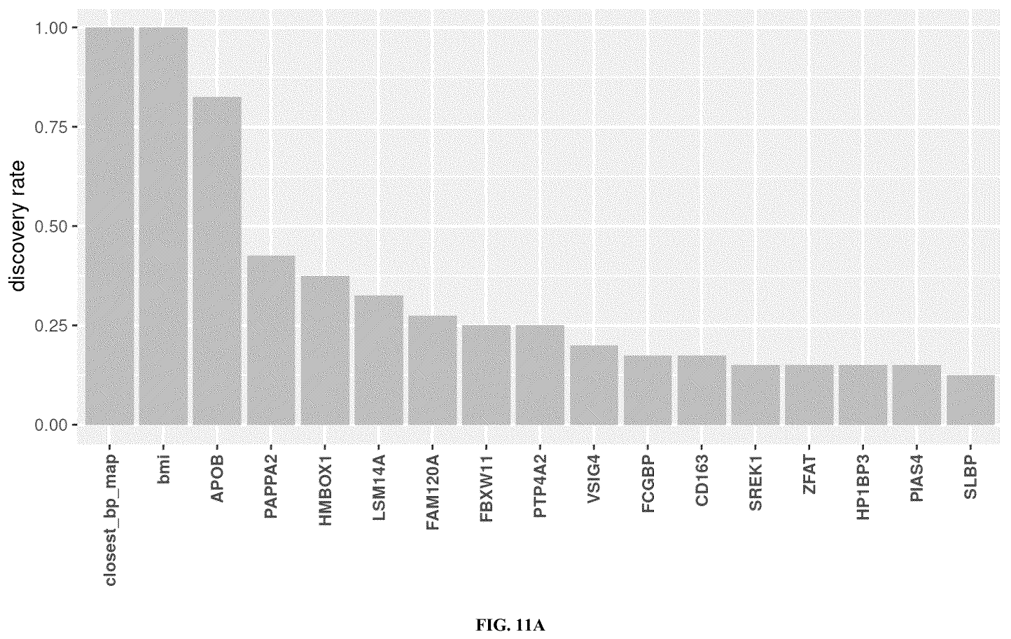

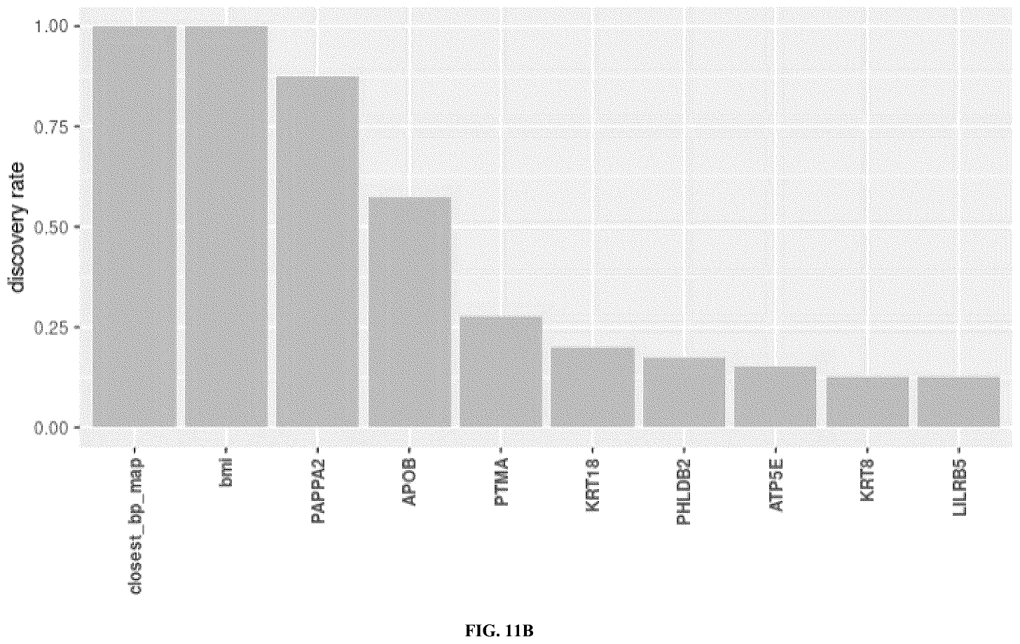

Additional analysis were performed to identify cfRNA variations in time of blood draw/collection associated with circadian clock, as shown in FIG. 10A. Samples were grouped by time of blood draw into morning (6 am to10 am, n=121), midday (10 am to2 pm, n=303), and afternoon (2 pm to6 pm, n=113). Differential gene expression (DGE) analyses were performed to elucidate the impact of time of day. A quasi-likelihood negative binomial generalized log-linear model was fitted to count data using an edgeR package (v. 3.38.1), and DGE were discovered with an empirical Bayes quasi-likelihood F-test (edgeR) in all 3 possible pairwise comparisons. When looking for DGE between the morning and afternoon groups, 5,729 genes, or 43% of all analyzed genes, were determined to be significantly differentially expressed (FIG. 10B). For morning vs midday groups, 4,278 genes (32% of all analyzed genes) were determined to be DEG; and for midday vs afternoon groups, 15 genes (0.1% of all analyzed genes) were determined to be DGE. The three sets have very high overlap, ranging from 73% to 87% (p<10-30). Further, among the genes with highest separation reported were associated with circadian rhythm (e.g., PER1, PER3, DDIT4, FKBP5, RBM3, SOCS1, BTG1, and ARHGEF10L).

Further, biological variations associated with subject BMI, fetal fraction, and gestation age at blood collection are effectively regressed using similar techniques to increase power of gene discovery.

### Example 6: Early Prediction of Preeclampsia and Molecular Subtypes of Preeclampsia from a 3,036-Person Prospective Trial Using cfRNA Profiling

Using systems and methods of the present disclosure, early differentially expressed molecular markers of preeclampsia were identified in maternal blood samples using a prospective cohort of 3,036 cases described in Example 5. All samples were processed using a unified experimental and computational NGS pipeline for cell-free (cfRNA) sequencing. Twin pregnancies, cases of spontaneous preterm birth, and or preterm delivery based on non-preeclampsia diagnosis were excluded.

The study design was performed as follows. Two approaches were used. In the first, the cohort with preeclampsia cases were grouped by severity of preeclampsia diagnoses with medically induced deliveries to reduce the risk of composite adverse maternal outcomes for women. The severe preterm preeclampsia cases from cohort were grouped by delivery at less than 38 or 37 weeks of gestation age, as shown in FIGS. 8 and 9, respectively. In the second, observed major clinical factors based on USPSTF were added as candidate features in performing feature discovery for preterm preeclampsia cases, as shown in FIG. 7.

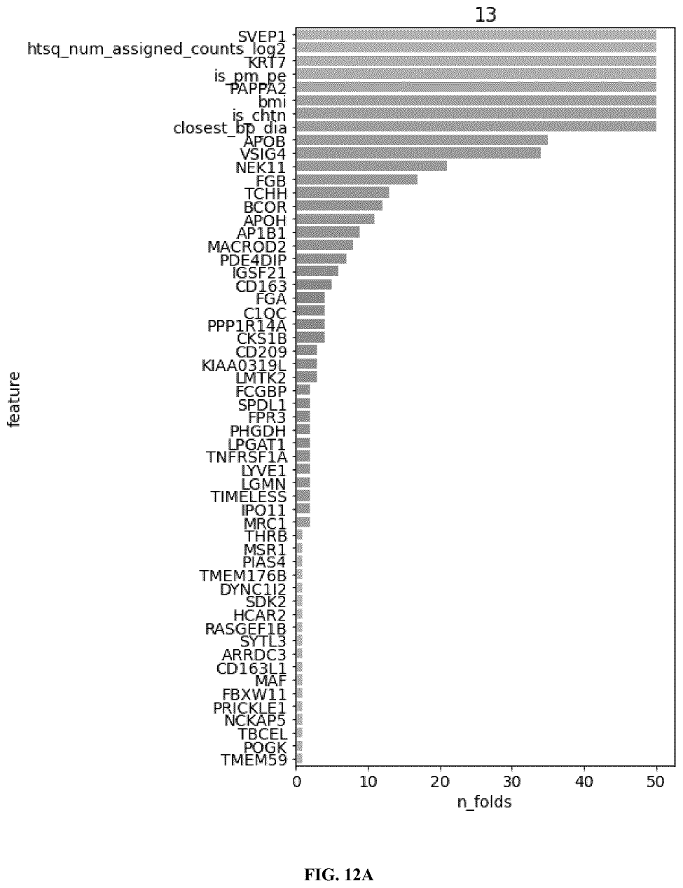

One approach to feature discovery is Sure Independence Screening, which ensures features that are orthogonal to each other to capture the biggest variation in the data. Using this method for the entire data set, a preterm preeclampsia signal was analyzed for delivering at less than 38 weeks gestational age. FIG. 11A and FIG. 11B show an example of the discovery rate for the genes associated with high risk developing preterm preeclampsia across a repeated cross-validation

Table 5 provides a listing of differentially expressed genes discovered by Sure Independence Screening, as being predictive for the molecular sub-type of preterm preeclampsia with medically prescribed delivery at less than 38 weeks. Similarly, Table 6 provides a listing of differentially expressed genes discovered by Sure Independence Screening, as being indicative for the molecular sub-type of preterm preeclampsia with medically prescribed delivery for delivery at less than 37 weeks of gestation age.

TABLE 5
Preterm preeclampsia differentially expressed genes
discovered by Sure Independence Screening as being
predictive of delivery at less than 38 weeks

| | | | | | |
|---|---|---|---|---|---|
| PAPPA2 | APOB | FBXW11 | PTP4A2 | SLBP | UBE2Q1 |
| SREK1 | PISA4 | ZNF148 | PTMA | KRT18 | PHLDB2 |
| ATP5E | KRT8 | LILRB5 | HMBOX1 | LSM14A | FAM120A |
| PTP4A2 | VSIG4 | FCGBP | CD163 | ZFAT | HP1BP3 |
| RPLP1 | RPS27 | MAU2 | MACROD2 | SELENOP | GID4 |
| NSRP1 | NEK11 | SVEP1 | PAPPA | MAFF | CD163L1 |
| KISS1 | FABP1 | ANGPT2 | APOH | | |

TABLE 6
Preterm preeclampsia differentially expressed genes
discovered by Sure Independence Screening as being
predictive of delivery at less than 37 weeks

| | | | | | |
|---|---|---|---|---|---|
| PAPPA2 | KRT18 | SELENOP | TOMM5 | PAPPA | ATP5E |
| APOB | KRT8 | GID4 | VAT1L | KISS1 | |

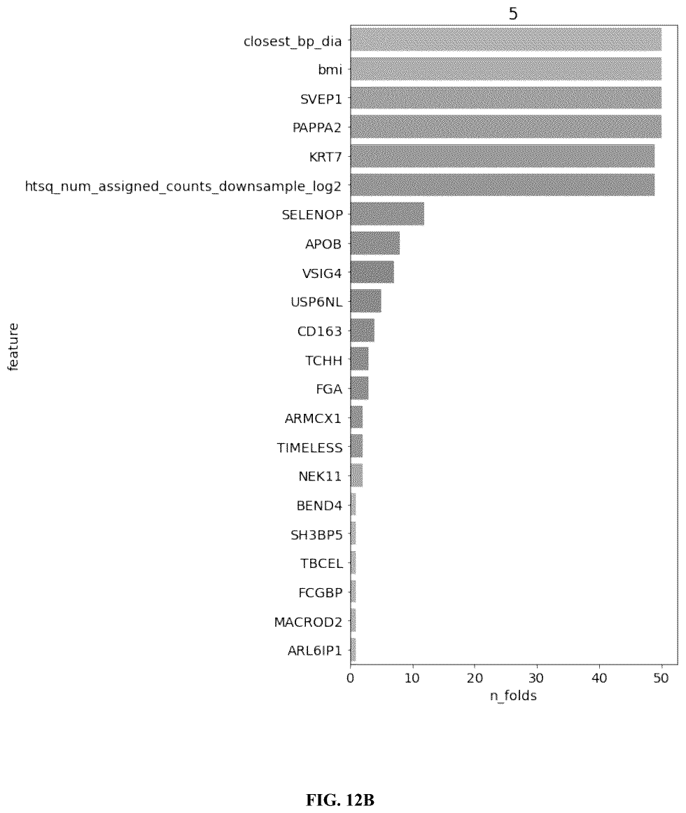

An alternative method for feature discovery is screening for differentially expressed genes in count spaces corrected for a variety of variables. Examples are correcting by BMI or total counts, or correcting by individual genes, such as KRT7 or SVEP1. Features are retained if they pass a multiple corrected p-value threshold and can be shown to add value to a model using the Aikake information criteria (AIC <−2). FIGS. 12A-12B depicts example outcomes for this type of modeling all gene markers and clinical factors discovered for preterm preeclampsia cases with delivery at less than 38 weeks or 37 weeks, respectively. Tables 7 and Table 8 provide a listing of all gene markers discovered by this approach for preterm preeclampsia cases with delivery at less than 38 weeks or 37 weeks, respectively.

TABLE 7
Preterm preeclampsia differentially expressed genes identified by
multiple space correction for deliveries at less than 38 weeks

| | | | | | |
|---|---|---|---|---|---|
| PAPPA2 | SVEP1 | KRT7 | VSIG4 | APOB | FGB |
| FCGBP | CD163 | TCHH | AXL | ETV5 | LYVE1 |
| IGSF21 | MACROD2 | FGG | C1QC | PPP1R14A | APOE |
| THRB | APOH | EFHD1 | TMEM176B | FABP1 | FPR3 |
| KISS1 | ALB | NEK11 | FGB | BCOR | AP1B1 |
| FGA | | | | | |

TABLE 8
Preterm preeclampsia genes identified by multiple
space correction for deliveries less than 37 weeks

| | | | | | |
|---|---|---|---|---|---|
| PAPPA2 | SELENOP | VSIG4 | APOB | CD163 | CD163L1 |
| TCHH | BCOR | FGA | USP6NL | SVEP1 | KRT7 |
| ARMCX1 | GATM | FPR3 | ETV5 | AXL | ZNF768 |
| TIMELESS | NEK11 | BEND4 | SH3BP5 | TBCEL | FCGBP |
| MACROD2 | ARL6IP1 | | | | |

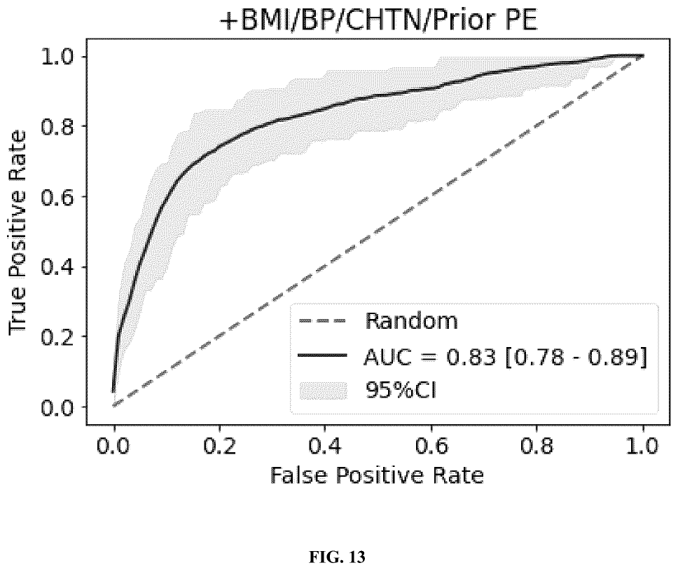

Modeling based on these features enabled the early prediction of preterm preeclampsia, yielding AUCs of up to 0.86 (for preterm preeclampsia cases with delivery at less than 36 weeks). FIG. 13 shows an example of an area-under-the-curve (AUC) for the ROC curve values with mean at 0.83 for ROC for a model predicting PE cases with deliveries at less than 37 weeks.

### Example 7: Early Prediction of Spontaneous Preterm Birth in a Prospective 3,036-Person Cohort Using Molecular Sub-Typing with cfRNA Profiling

Using systems and methods of the present disclosure, early differentially expressed molecular markers of preterm birth (PTB) were identified in maternal blood samples from a prospective cohort of 3,036 participants described in Example 5. All samples were processed using a unified experimental and computational NGS pipeline for cell-free (cfRNA) sequencing. Twin pregnancies, preterm birth cases of medically induced delivery based on preeclampsia diagnosis, and or other medically induces deliveries were excluded.

To identify differentially expressed gene markers for spontaneous preterm birth, two types of differential gene expression analyses were performed for two different molecular subtypes of spontaneous preterm birth, defined as either delivery at less than 35 weeks or delivery less than 37 weeks.

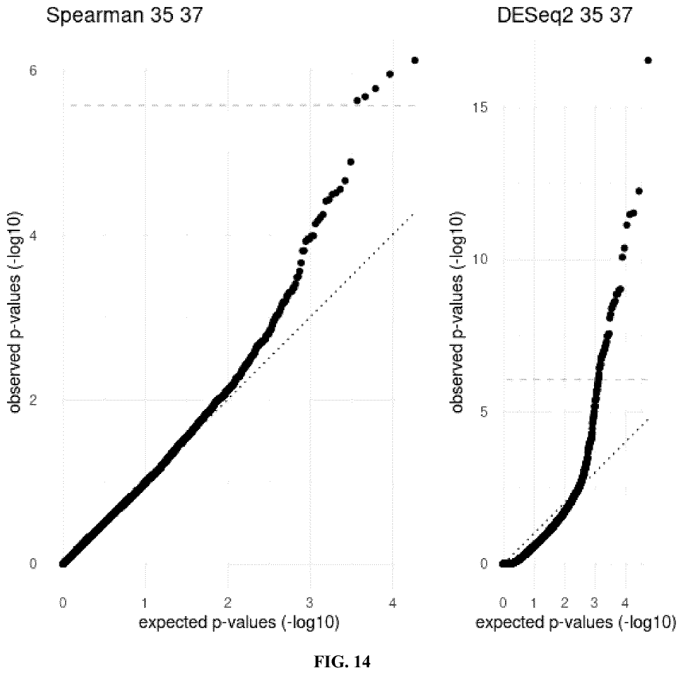

First, Spearman and DESeq2 differential gene expression analyses were performed for 55 spontaneous preterm birth cases with delivery before 35 weeks of gestational age and 2,899 full term birth control cases with delivery after 37 weeks of gestational age. FIG. 14 shows a quantile-quantile (QQ) plot for a differential gene expression signal for differentially expressed genes in pre-term birth cases with delivery before 35 weeks of gestational age. Table 9 shows a set of top 5 differentially expressed genes by Spearman ranked analyses for predicting spontaneous preterm birth cases with delivery earlier than 35 weeks of gestation.

TABLE 9
Set of top 5 differentially expressed genes that are predictive
for spontaneous preterm birth cases with delivery earlier
than 35 weeks using Spearman ranked analyses

| Index | Gene | P-value |
|---|---|---|
| 1 | ZNF812P | 0.01418108 |
| 2 | FPR3 | 0.020841643 |
| 3 | LILRB5 | 0.031239504 |
| 4 | CLEC9A | 0.039139074 |
| 5 | ETV5 | 0.043737094 |

Table 10 shows a set of top 43 differentially expressed genes by DESeq2 differential gene expression analyses for predicting spontaneous preterm birth cases with delivery earlier than 35 weeks of gestation.

TABLE 10
Set of top 43 differentially expressed genes that are predictive
for spontaneous preterm birth cases with delivery earlier
than 35 weeks using DESeq2 differential gene expression

| Index | Gene | P-value |
|---|---|---|
| 1 | LYVE1 | 1.66E−12 |
| 2 | TCHH | 3.24E−08 |
| 3 | CD163 | 1.68E−07 |
| 4 | DUSP27 | 1.91E−07 |
| 5 | VSIG4 | 4.21E−07 |
| 6 | MRC1 | 2.41E−06 |
| 7 | GPR82 | 4.81E−06 |
| 8 | GPR34 | 5.36E−05 |
| 9 | MSR1 | 5.74E−05 |
| 10 | RNASE1 | 7.74E−05 |
| 11 | TREM2 | 7.86E−05 |
| 12 | FPR3 | 1.31E−04 |
| 13 | FCGBP | 1.44E−04 |
| 14 | MERTK | 1.71E−04 |
| 15 | STAB1 | 2.13E−04 |
| 16 | LILRB5 | 2.29E−04 |
| 17 | CADM1 | 3.60E−04 |
| 18 | SELENOP | 4.75E−04 |
| 19 | NCKAP5 | 0.00155276 |
| 20 | C1QC | 0.00160502 |
| 21 | C1QB | 0.00182701 |
| 22 | DBNDD2 | 0.00187367 |
| 23 | MMP2 | 0.00287475 |
| 24 | AXL | 0.00317878 |
| 25 | GATM | 0.00387293 |
| 26 | FOLR2 | 0.00412276 |
| 27 | ZNF812P | 0.00530928 |
| 28 | ETV5 | 0.00546725 |
| 29 | MTCO1P12 | 0.00637143 |
| 30 | CLEC9A | 0.00696217 |
| 31 | MMP14 | 0.00759331 |
| 32 | CD209 | 0.0080383 |
| 33 | ZFHX3 | 0.00868013 |
| 34 | EPS8 | 0.00933179 |
| 35 | SLCO2B1 | 0.01086637 |
| 36 | OLFML3 | 0.01562314 |
| 37 | CD276 | 0.0170661 |
| 38 | CABLES1 | 0.01958957 |
| 39 | SDC3 | 0.01977068 |
| 40 | MAF | 0.02108906 |
| 41 | RGL1 | 0.03271522 |
| 42 | SPRED1 | 0.04326907 |
| 43 | UNC13C | 0.04952278 |

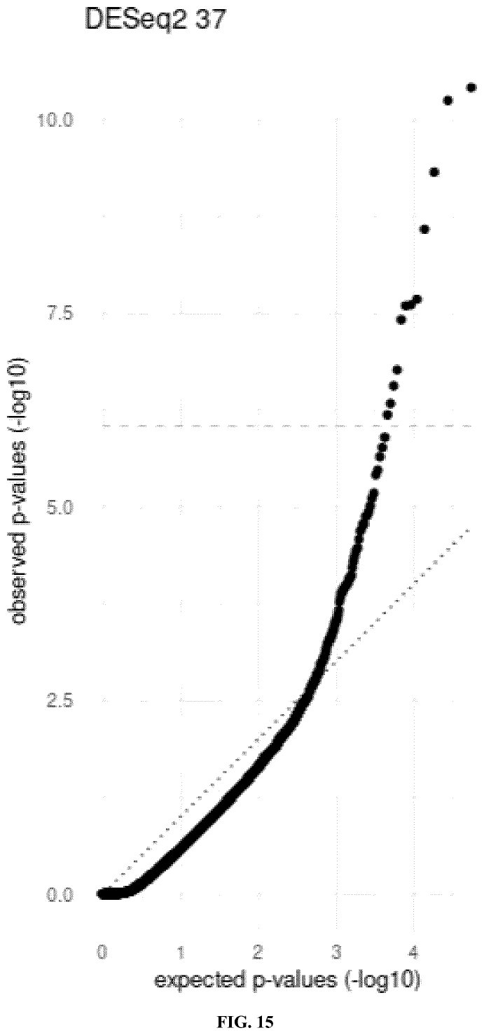

Similar analyses with DESeq2 differential gene expression were performed for 135 spontaneous preterm birth cases with spontaneous birth before 37 weeks of gestational age and 2,899 full term birth control cases with delivery after 37 weeks of gestational age. FIG. 15 shows a quantile-quantile (QQ) plot for a differential gene expression signal for differentially expressed genes in pre-term birth cases with delivery before 37 weeks of gestational age.

Table 11 shows a set of top 12 differentially expressed genes by DESeq2 differential gene expression analyses for predicting spontaneous preterm birth cases with delivery earlier than 37 weeks of gestation.

TABLE 11
Set of 12 top differentially expressed genes that are predictive
for spontaneous preterm birth cases with delivery earlier
than 37 weeks using DESeq2 differential gene expression

| Index | Gene | P-value |
|---|---|---|
| 1 | TCHH | 2.15E−06 |
| 2 | LYVE1 | 3.15E−06 |
| 3 | GPR82 | 2.65E−05 |
| 4 | CD163 | 1.45E−04 |
| 5 | SAA2 | 0.001162 |
| 6 | DUSP27 | 0.001376 |
| 7 | CSRP3 | 0.00141 |
| 8 | VSIG4 | 0.002136 |
| 9 | GPR34 | 0.009453 |
| 10 | MRC1 | 0.015219 |
| 11 | FPR3 | 0.025791 |
| 12 | ADGRG7 | 0.036084 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-72. (canceled)

73. A method for treating a pregnant subject for a pregnancy complication, comprising:
(a) obtaining a cell-free biological sample from said pregnant subject, wherein said pregnant subject has a clinical history that is indicative of an elevated risk of said pregnancy complication, and wherein said pregnant subject is asymptomatic for said pregnancy complication;
(b) assaying nucleic acid molecules obtained from said cell-free biological sample to determine at least one ribonucleic acid (RNA) level of at least one pregnancy-associated gene, wherein said at least one pregnancy-associated gene is differentially expressed in a first population of pregnant subjects with a pregnancy involving said pregnancy complication as compared to a second population of pregnant subjects without a pregnancy involving said pregnancy complication;
(c) computer processing said at least one RNA level of said at least one pregnancy-associated gene determined in (b) (i) against at least one reference RNA level of said at least one pregnancy-associated gene or (ii) with a trained machine learning algorithm;
(d) determining, based at least in part on said computer processing in (c), that said pregnant subject has a presence or elevated risk of a molecular sub-type of said pregnancy-related complication; and
(e) administering a treatment to said pregnant subject for said molecular sub-type of said pregnancy complication, based at least in part on said determining in (d).

74. The method of claim 73, wherein said pregnancy complication is selected from the group consisting of pre-term birth, a pregnancy-related hypertensive disorder, preeclampsia, eclampsia, gestational diabetes, a congenital disorder of a fetus of said pregnant subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine growth restriction, fetal growth restriction, macrosomia, a neonatal condition, and an abnormal fetal development.

75. The method of claim 73, wherein said pregnancy complication comprises pre-term birth.

76. The method of claim 75, wherein said molecular subtype of pre-term birth is selected from the group consisting of history of prior pre-term birth, spontaneous pre-term birth, ethnicity specific pre-term birth risk, and pre-term premature rupture of membrane (PPROM).

77. The method of claim 76, wherein said molecular subtype of pre-term birth comprises spontaneous pre-term birth.

78. The method of claim 77, wherein said at least one pregnancy-associated gene associated with spontaneous pre-term birth is selected from the group consisting of genes listed in Table 1, genes listed in Table 2, genes listed in Table 3, genes corresponding to a pathway listed in Table 4, genes listed in Table 9, genes listed in Table 10, and genes listed in Table 11.

79. The method of claim 75, wherein said treatment comprises a drug, a supplement, a lifestyle recommendation, a cervical cerclage, a cervical pessary, or electrical contraction inhibition.

80. The method of claim 79, wherein said drug is selected from the group consisting of progesterone, erythromycin, a tocolytic medication, a corticosteroid, a vaginal flora, and an antioxidant.

81. The method of claim 73, wherein said pregnancy complication comprises preeclampsia.

82. The method of claim 81, wherein said molecular subtype of preeclampsia is selected from the group consisting of history of chronic or pre-existing hypertension, presence or history of gestational hypertension, presence or history of mild preeclampsia, presence or history of severe preeclampsia, presence or history of eclampsia, and presence or history of HELLP (Hemolysis, Elevated Liver enzymes and Low Platelets) syndrome.

83. The method of claim 82, wherein said molecular subtype of preeclampsia comprises pre-term preeclampsia.

84. The method of claim 83, wherein said at least one pregnancy-associated gene associated with pre-term preeclampsia is selected from the group consisting of genes listed in Table 5, genes listed in Table 6, genes listed in Table 7, and genes listed in Table 8.

85. The method of claim 81, wherein said treatment comprises a drug selected from the group consisting of aspirin, progesterone, magnesium sulfate, a cholesterol medication, a heartburn medication, an angiotensin II receptor antagonist, a calcium channel blocker, a diabetes medication, and an erectile dysfunction medication.

86. The method of claim 73, wherein said pregnancy complication comprises gestational diabetes.

87. The method of claim 86, wherein said at least one pregnancy-associated gene associated with gestational diabetes is selected from the group consisting of PDK4, CSH1, PLAC4, TBCEL, and FBXO7.

88. The method of claim 86, wherein said treatment comprises a drug, a supplement, a lifestyle recommendation, a cervical cerclage, a cervical pessary, or electrical contraction inhibition.

89. The method of claim 73, wherein said cell-free biological sample is selected from the group consisting of plasma, serum, urine, saliva, amniotic fluid, and derivatives thereof.

90. The method of claim 89, wherein said cell-free biological sample is plasma.

91. The method of claim 73, further comprising fractionating a whole blood sample of said pregnant subject to obtain said cell-free biological sample.

92. The method of claim 73, wherein said trained machine learning algorithm is trained using a first set of independent training samples associated with a presence or elevated risk of said pregnancy complication and a second set of independent training samples associated with an absence or no elevated risk of said pregnancy complication.

93. The method of claim 73, further comprising extracting RNA molecules from said cell-free biological sample, and sequencing said RNA molecules or derivatives thereof to generate a set of sequencing reads.

94. The method of claim 93, wherein said sequencing comprises massively parallel sequencing.

95. The method of claim 93, wherein said sequencing comprises nucleic acid amplification.

96. The method of claim 95, wherein said nucleic acid amplification comprises polymerase chain reaction (PCR).

97. The method of claim 93, wherein said sequencing comprises reverse transcription (RT).

98. The method of claim 73, further comprising using nucleic acid primers or probes to selectively enrich said nucleic acid molecules corresponding to a panel of one or more genomic loci, wherein said nucleic acid primers or probes have sequence complementarity with nucleic acid sequences of said panel of said one or more genomic loci.

99. The method of claim 73, wherein said trained machine learning algorithm comprises a deep learning algorithm, a support vector machine (SVM), a neural network, a Random Forest, a linear regression model, a logistic regression model, or an ANOVA (analysis of variance) model.

100. The method of claim 73, further comprising monitoring said presence or elevated risk of said molecular sub-type of said pregnancy complication, wherein said monitoring comprises assessing said presence or elevated risk of said molecular sub-type of said pregnancy complication of said pregnant subject at a plurality of time points.
